(19) United States
(12) Patent Application Publication
(10) Pub. No.: US 2026/0224386 A1
(43) Pub. Date: Aug. 6, 2026

(54) A CERVICAL COLLAR AND ASSOCIATED METHODS OF USE

(71) Applicant: DeRoyal Industries, Inc., Powell, TN (US)

(72) Inventors: Walter Cleveland Cowart, Blaine, TN (US); Sarah O. Davis, Knoxville, TN (US); Nicholas John Poker, Knoxville, TN (US); John Connor Brown, Knoxville, TN (US); Charles Joseph French, III, Knoxville, TN (US); Rebecca Laughon, Powell, TN (US); Dhanvin Sunil Desai, Knoxville, TN (US)

(21) Appl. No.: 19/152,401

(22) PCT Filed: Jan. 30, 2024
(86) PCT No.: PCT/US2024/013590

§ 371 Date: Jul. 30, 2025

Related U.S. Application Data
(63) Continuation-in-part of application No. 18/102,889, filed on Jan. 30, 2023.

(51) Int. Cl. A61F 5/055

(52) U.S. Cl. CPC A61F 5/055

(57) ABSTRACT

An adjustable cervical collar containing a lower support arm and a pivotable upper chin support arm coupled to the lower support on opposing sides. The lower support arm includes a lower support arm cover and a lower support arm locking mechanism. The lower support arm cover can comprise a lower support arm inner shell and a lower support arm outer shell. The pivotable upper chin support arm is coupled to the lower support on opposing sides; the upper chin support arm includes a pair of chin support arm outer components and a chin support arm center component.

### CROSS-REFERENCE TO RELATED APPLICATIONS

This application is a US National Stage Entry of PCT/US24/13590, entitled “A CERVICAL COLLAR AND ASSOCIATED METHODS OF USE” filed Jan. 30, 2024, which is a continuation-in-part of U.S. patent application Ser. No. 18/102,889, entitled “CERVICAL COLLAR” filed Jan. 30, 2023, the contents of which are incorporated herein by reference in its entirety.

### TECHNICAL FIELD

This application relates in general to an article of manufacture for providing a healthcare aid, and more specifically, to an article of manufacture providing an adjustable cervical collar.

### BACKGROUND

Cervical collars are useful healthcare aid devices that help a user support the neck and head while preventing the wearer from moving the head from a safe and comfortable position, The cervical collar typically is worn when the user has suffered an injury to the head, neck, and/or shoulders that will benefit from keeping the head and neck in a stationary position while the condition heals. Immobilizing a head and neck into a single position can become tiring and uncomfortable for the wearer when the cervical collar is ill-fitting and not properly supporting the head and/or neck. Adjusting a cervical collar and its supporting components to fit users of different sizes and positioning the cervical collar into a variety of configurations to support users having different injuries may be challenging.

Therefore, a need exists for an article of manufacture providing an adjustable cervical collar. The present invention attempts to address the limitations and deficiencies in prior solutions according to the principles and example embodiments disclosed herein.

### SUMMARY

In accordance with the present invention, the above and other problems are solved by providing an article of manufacture for an adjustable cervical collar according to the principles and example embodiments disclosed herein.

In one embodiment, the present invention is an article of manufacture providing an adjustable cervical collar. The adjustable cervical collar contains a lower support arm having opposing ends and a pivotable upper chin support arm coupled to the lower support arm on opposing sides. In embodiments, the lower support arm includes a lower support arm cover and a lower support arm locking mechanism. The lower support arm cover can comprise a lower support arm inner shell and a lower support arm outer shell. The pivotable upper chin support arm can include a pair of chin support arm outer components and a chin support arm center component.

In another aspect, the invention relates to methods of adjusting the cervical collar according to the embodiments disclosed herein.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

Referring now to the drawings in which like reference numbers represent corresponding parts throughout:

FIGS. 1a-c illustrate example embodiments of an article of manufacture providing an adjustable cervical collar according to the present invention.

FIGS. 2a-b illustrate various embodiments of an article of manufacture providing an adjustable cervical collar according to the present invention.

FIGS. 3a-b illustrate a rear support member coupling to a front arm assembly of an article of manufacture providing an adjustable cervical collar according to the present invention.

FIGS. 4a-e illustrate various front views of example embodiments of an article of manufacture providing an adjustable cervical collar according to the present invention.

FIGS. 5a-d illustrate various side views of coupling a lower support arm and an upper chin support arm of an article of manufacture providing an adjustable cervical collar according to the present invention.

FIGS. 6a-d illustrate various top and bottom views of an article of manufacture providing an adjustable cervical collar according to the present invention.

FIGS. 7a-d illustrate various views of coupling members between a lower support arm and an upper chin support arm of an article of manufacture providing an adjustable cervical collar according to the present invention.

FIGS. 8a-f illustrate various views of a lower support arm of an article of manufacture providing an adjustable cervical collar according to the present invention.

FIGS. 9a-h illustrate various views of a rear support member of an article of manufacture providing an adjustable cervical collar according to the present invention.

FIGS. 10a-c illustrate various views of a lower support arm with a pair of position locking mechanisms of an article of manufacture providing an adjustable cervical collar according to the present invention.

FIGS. 11a-f illustrate various views of components of each of the pair of position locking mechanisms of an article of manufacture providing an adjustable cervical collar according to the present invention.

FIGS. 12a-e illustrate open and closed configurations of each of the pair of position locking mechanisms of an article of manufacture providing an adjustable cervical collar according to the present invention.

FIGS. 13a-c illustrate multiple views of a lower support arm padding used with the lower support arm of an article of manufacture providing an adjustable cervical collar according to the present invention.

FIGS. 14a-c illustrate multiple views of a rear support member padding used with the rear support member of an article of manufacture providing an adjustable cervical collar according to the present invention.

FIGS. 15a-b illustrate multiple views of an upper chin support arm padding used with the upper chin support arm of an article of manufacture providing an adjustable cervical collar according to the present invention.

FIG. 16a provides a top, front perspective view of an adjustable cervical collar according to an alternate embodiment of the present invention.

FIG. 16b illustrates a bottom, rear perspective view of the adjustable cervical collar of the FIG. 16a embodiment.

FIG. 17a shows a top, front perspective view of a front assembly of the cervical collar from the embodiment shown in FIG. 16a.

FIG. 17b provides a bottom, rear perspective view of front assembly of FIG. 17a.

FIG. 17c shows a top plan view of the front assembly of FIG. 17a.

FIG. 17d provides a side elevation view of the front assembly of FIG. 17a.

FIG. 17e illustrates a front elevation view of the front assembly of FIG. 17a.

FIG. 18a illustrates a front elevation view of the lower support arm from the front assembly of FIG. 17a. The lower support arm outer shell is shown in phantom and the lower support arm inner shell is removed to reveal the lower support arm locking mechanism as disposed within the lower support arm.

FIG. 18b shows a front, bottom perspective view of the lower support arm of the front assembly of FIG. 17a. The lower support arm outer shell is shown in phantom and the lower support arm inner shell is removed to reveal the lower support arm locking mechanism as disposed within the lower support arm.

FIG. 19 illustrates an exploded, top perspective view of the right-side lower support arm locking mechanism of FIG. 18a. The right-side lower support arm locking mechanism is shown isolated from the lower support arm.

FIG. 20a provides a top, side perspective view of the locking mechanism of the FIG. 18a embodiment. The locking mechanism is shown isolated from the lower support arm, and the right locking mechanism release button is shown in phantom.

FIG. 20b provides a top, side perspective partial view of the locking mechanism of the FIG. 18a embodiment. In this view, the partial locking mechanism is shown isolated from the lower support arm, and the right spring-loaded sliding lock is removed from the locking mechanism to provide an unobstructed view of the right anchored locking tooth component. In addition, the right locking mechanism release button and left elongated connector are shown in phantom.

FIG. 20c provides a top, side perspective partial view of the locking mechanism of the FIG. 18a embodiment. In this view, the partial locking mechanism is shown isolated from the lower support arm. The right spring-loaded sliding lock and the right anchored locking tooth component are removed from the locking mechanism to provide an unobstructed view of the right toothed sliding height adjustment piece and a clearer view of the right elongated connector. The left spring-loaded sliding lock is also removed. In addition, the left elongated connector is shown in phantom.

FIG. 20d provides a bottom, side perspective partial view of a portion of the left locking mechanism of the FIG. 18a embodiment. In this view, the partial locking mechanism is shown isolated from the lower support arm. The left anchored locking tooth component is shown in phantom.

### DETAILED DESCRIPTION

This application relates in general to an article of manufacture for providing a healthcare aid, and more specifically, to an article of manufacture providing an adjustable cervical collar according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps, or components, but do not preclude the presence or addition of one or more other features, steps, or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

The terms “individual” and “user” refer to an entity, e.g., a human, using an article of manufacture providing an adjustable cervical collar according to the present invention. The term “user” herein refers to one or more users.

The terms “invention” or “present invention” refer to the invention as described over various aspects and embodiments in the present patent application entitled “A Cervical Collar.” The term “invention” may be used interchangeably with “the collar.”

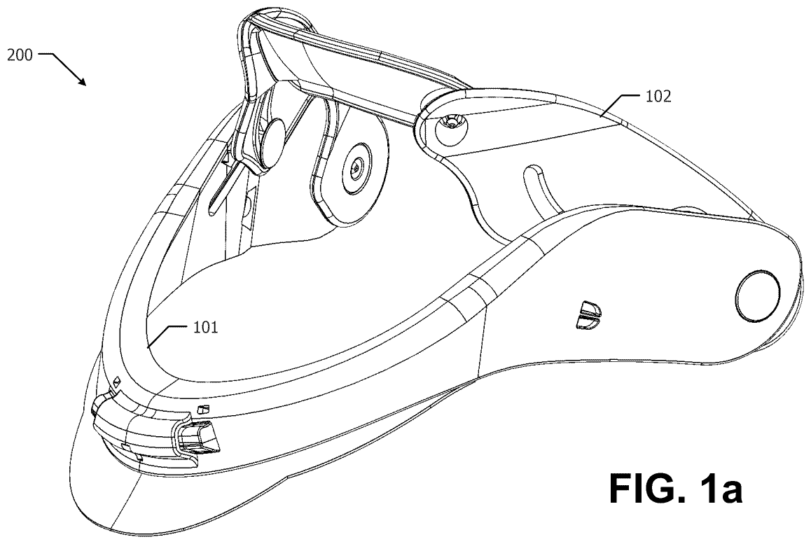

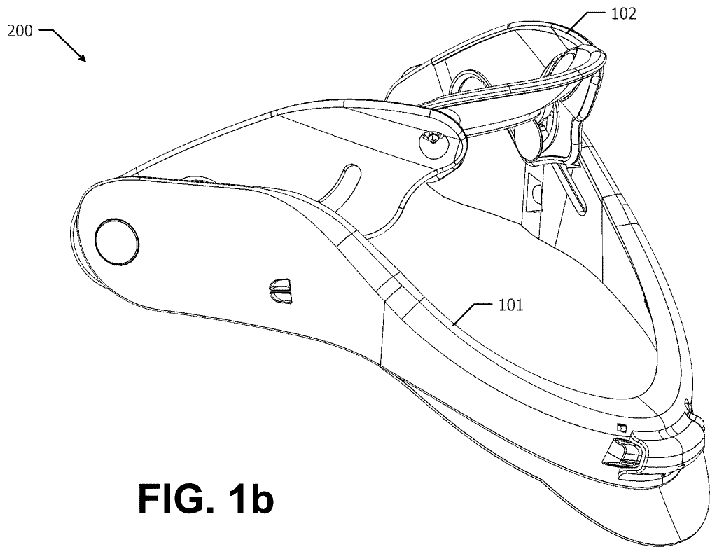

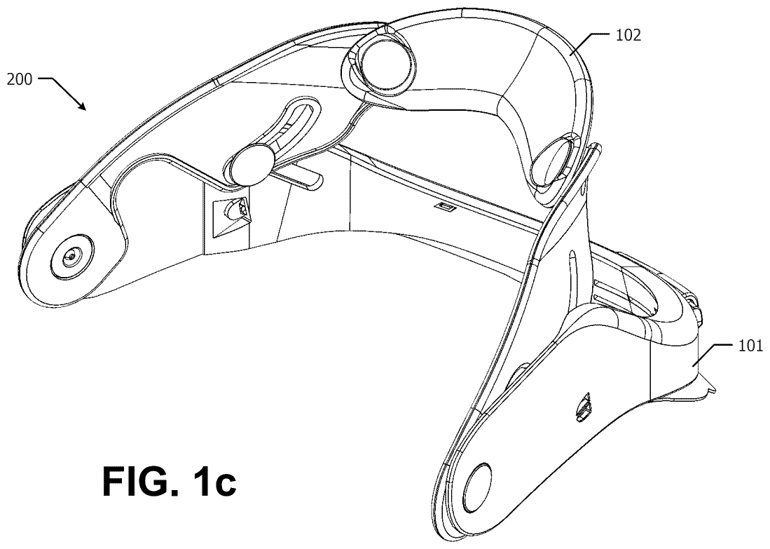

In general, the present disclosure relates to an article of manufacture providing an adjustable cervical collar. FIGS. 1a-c illustrate example embodiments of an article of manufacture providing an adjustable cervical collar according one embodiment of the present invention. FIG. 1a shows a right perspective view of a front assembly 200 of the adjustable cervical collar 100. The front part of the cervical collar 100 includes a lower support arm 101 coupled to a pivotable upper chin support arm 102. Both lower support arm 101 and upper chin support arm 102 are U-shaped arms that are coupled together at the outer ends of the arms. The upper chin support arm 102 is positioned above the lower support arm 101 when worn by a user.

The lower support arm 101 is worn in a downward configuration in which the lower support arm 101 rests against the user's chest with the pivot point of the two arms being positioned above a user's shoulders. The upper chin support arm 102 typically extends outward to position a top portion of the upper chin support arm 102 just below the user's chin and jaw. The upper chin support arm 102 and lower support arm 101 are lockable to configure the position of the lower support arm 101 relative to the upper chin support arm 102 to remain in a user-defined configuration. With the lower support arm 101 resting upon the user's chest, the user may rest his or her chin on top of the upper chin support arm 102, allowing the front assembly 200 to support the weight of the user's head.

FIG. 1b shows a left perspective view of the adjustable cervical collar. The cervical collar 100 is symmetrical with each side of the lower support arm 101 and upper chin support arm 102 being identical. At the center of the lower support arm 101, a pair of locking mechanism release buttons 504a-b are positioned to be easily accessible to the user. When the user depresses the locking mechanism release buttons 504a-b, a locking mechanism on each side of the lower support arm 101 unlocks to permit the adjustment of the position of the lower support arm 101 relative to the upper chin support arm 102.

When the lower support arm 101 and upper chin support arm 102 are in a desired configuration, the user releases the locking mechanism release buttons 504a-b to relock the locking mechanism to restrain the configuration of the lower support arm 101 and upper chin support arm 102.

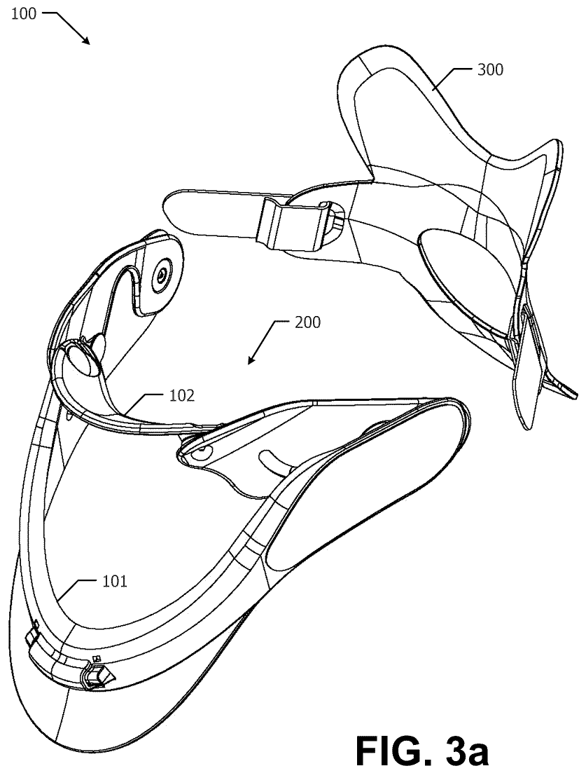

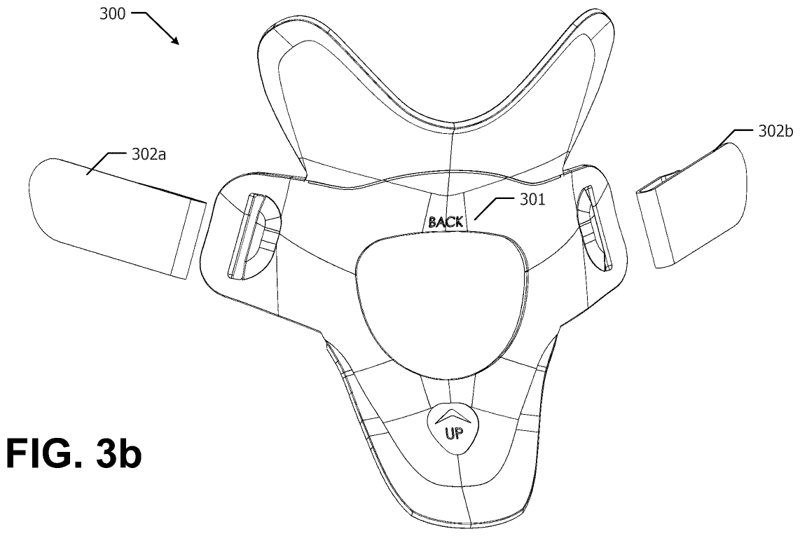

FIG. 1c shows a rear perspective view of the adjustable cervical collar. The rear side of the cervical collar 100 shows the inside surfaces of the lower support arm 101 and upper chin support arm 102 that are placed about the user's head and neck to place the cervical collar 100 in position. The lower support arm 101 and upper chin support arm 102 form a front assembly 200. The cervical collar 100 is secured to the user by attaching a rear support member 300 shown in FIGS. 3a-b to the front assembly 200. The rear support member 300 is tightened to the front assembly 200 using straps to secure the cervical collar 100 in place around the user's neck.

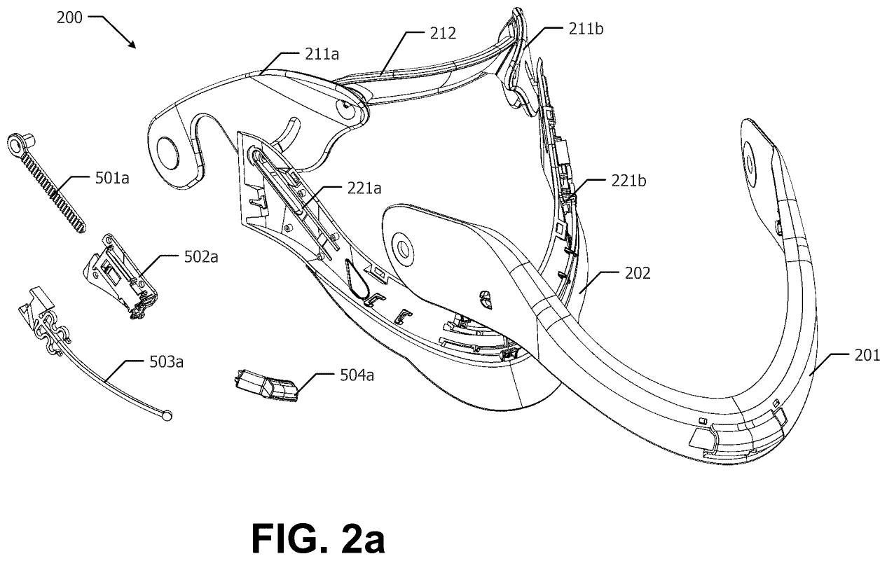

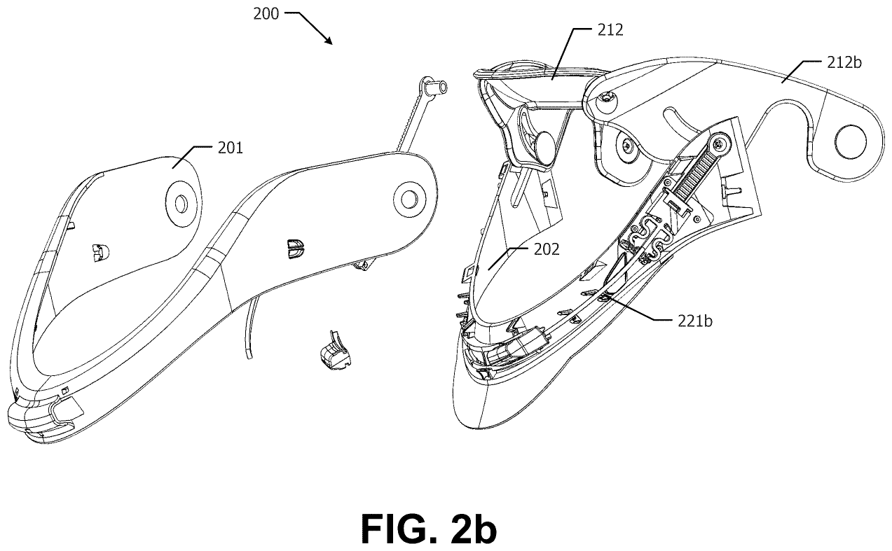

FIGS. 2a-b illustrate various embodiments of an article of manufacture providing an adjustable cervical collar according to the present invention. The lower support arm 101 is shown having a lower support arm outer shell 201 coupled over a lower support arm inner shell 202. A pair of lower support arm locking mechanisms 221a-b is coupled to both sides of the lower support arm inner shell 202 to lock and unlock the lower support arm 101 and upper chin support arm 102 in a desired configuration. The lower support arm outer shell 201 encases the lower support arm locking mechanisms 221a-b and the lower support arm inner shell 202 within the lower support arm 101. A lower end of each of the lower support arm locking mechanisms 221a-b connects each of the locking mechanism release buttons 504a-b to the pivot point between the lower support arm 101 and the upper chin support arm 102 to lock and unlock the lower support arm locking mechanisms 221a-b.

The locking mechanism release buttons 504a-b extend out of the lower support arm outer shell 201 adjacent to each other allowing the user to depress both of the locking mechanism release buttons 504a-b at the same time permitting the user to simultaneously pivot the lower support arm 101 from the upper chin support arm 102 on both sides thereby making the reconfiguration of the cervical collar 100 straightforward.

FIG. 2b shows an exploded view of the left side of the lower support arm. The alternate side of the lower support arm 101 is identical to the corresponding side of the lower support arm 101 having a similar lower support arm locking mechanism 221b under the lower support arm outer shell 201.

FIGS. 3a-b illustrate a rear support member coupling to a front arm assembly of an article of manufacture providing an adjustable cervical collar according to the present invention. FIG. 3a shows the cervical collar 100. The cervical collar 100 is shown in a configuration allowing the rear support member 300 to be coupled to the front assembly 200 by a pair of rear support member attachment loop straps 302a-b. The rear support member 300 is connected to lower support arm 101 with loop straps and adhesive hook.

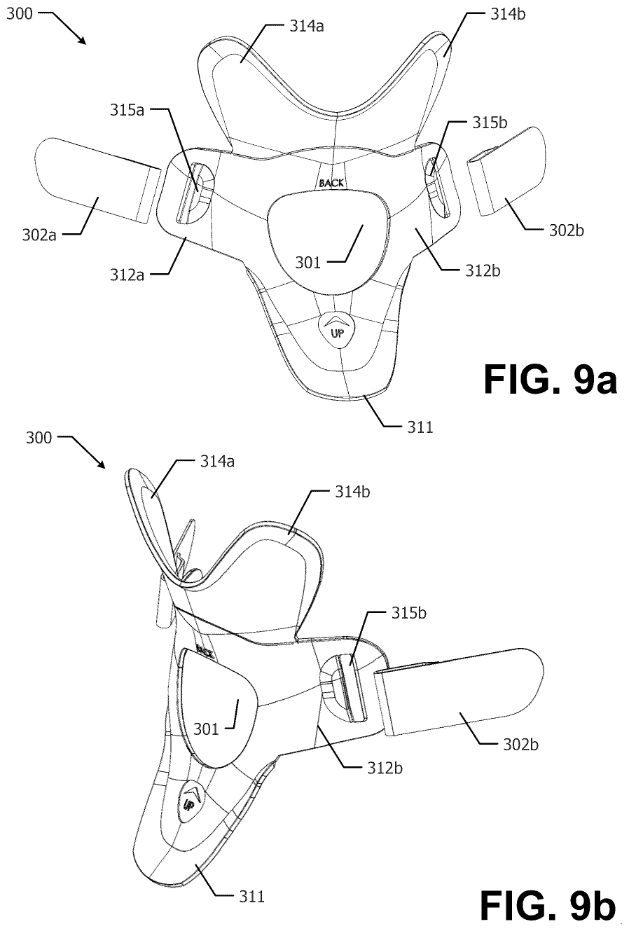

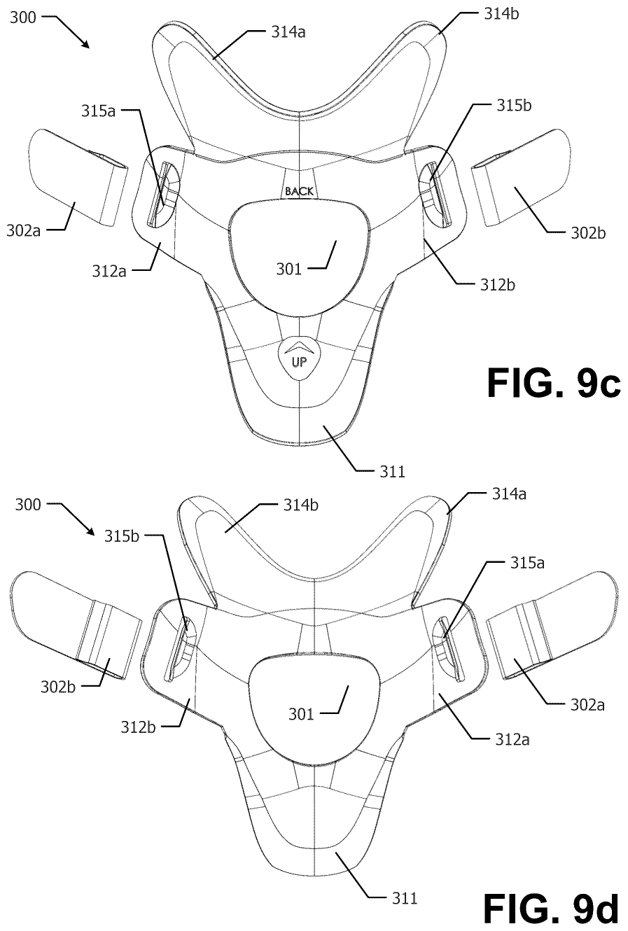

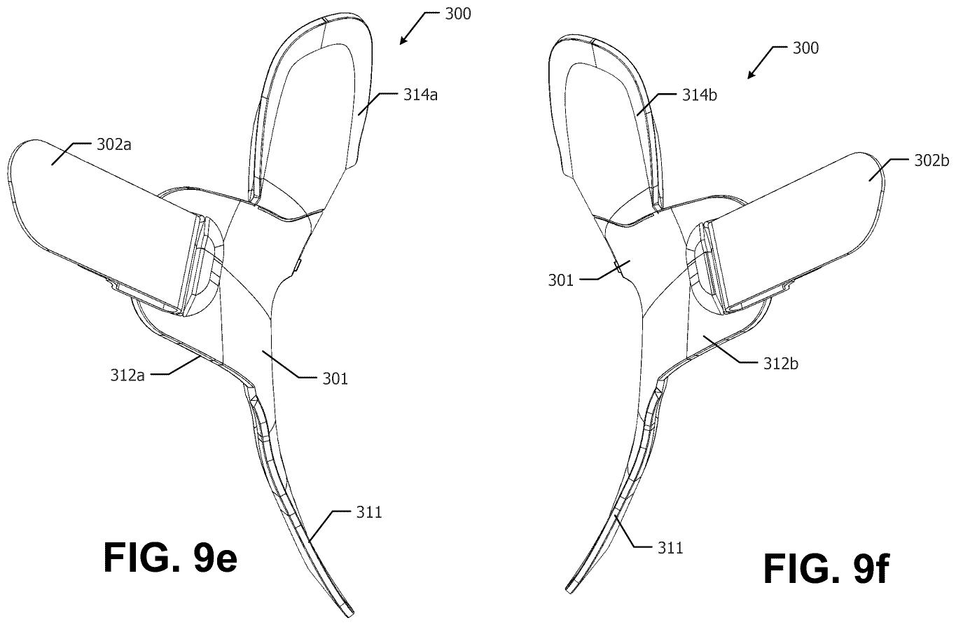

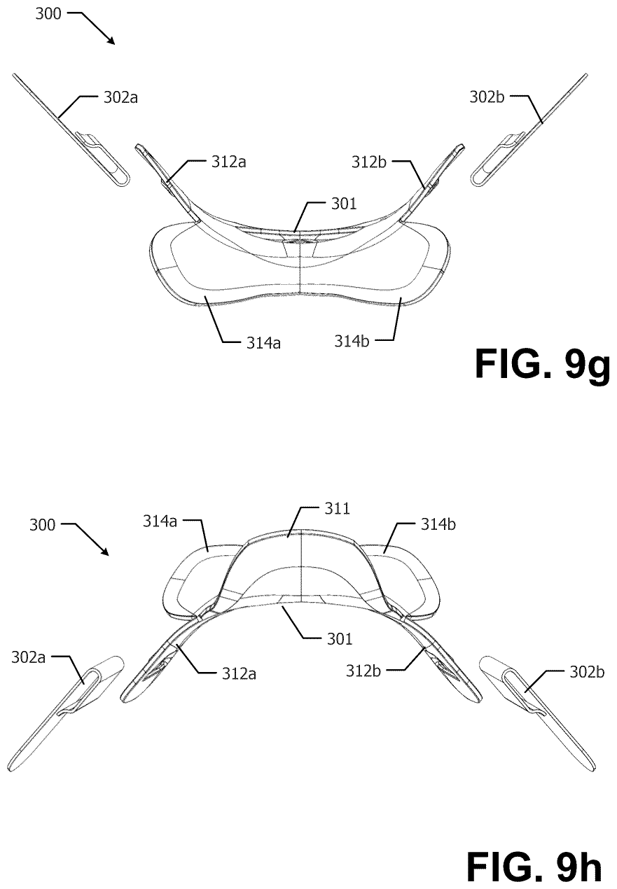

FIG. 3b shows an exploded view of the rear support member. The rear support member 300 includes a rear support member frame component 301 and rear support member attachment loop straps 302a-b. The rear support member 300 and its rear support member frame component 301 and rear support member attachment loop straps 302a-b are described in more detail below in reference to FIGS. 9a-f.

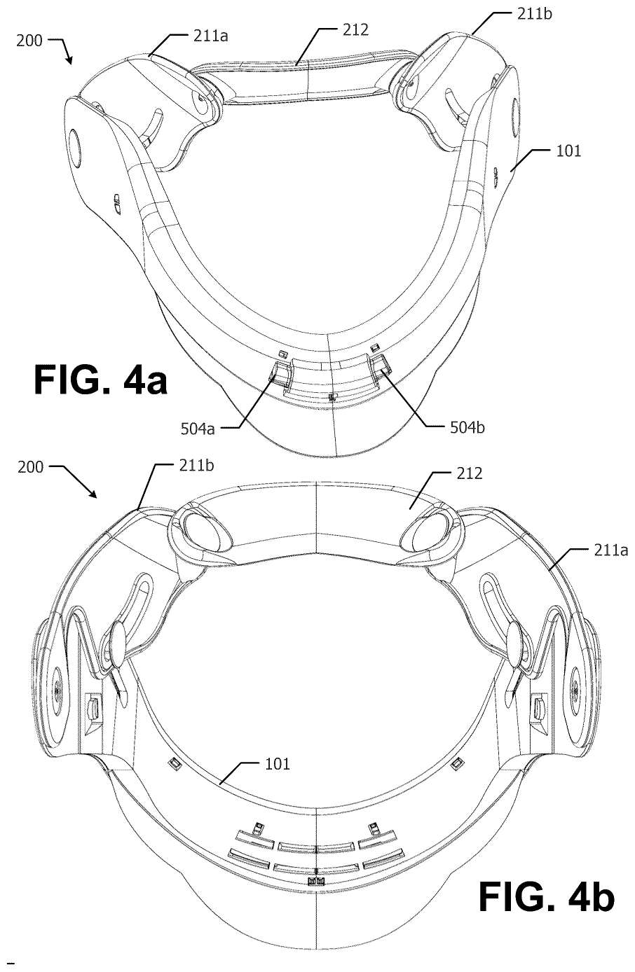

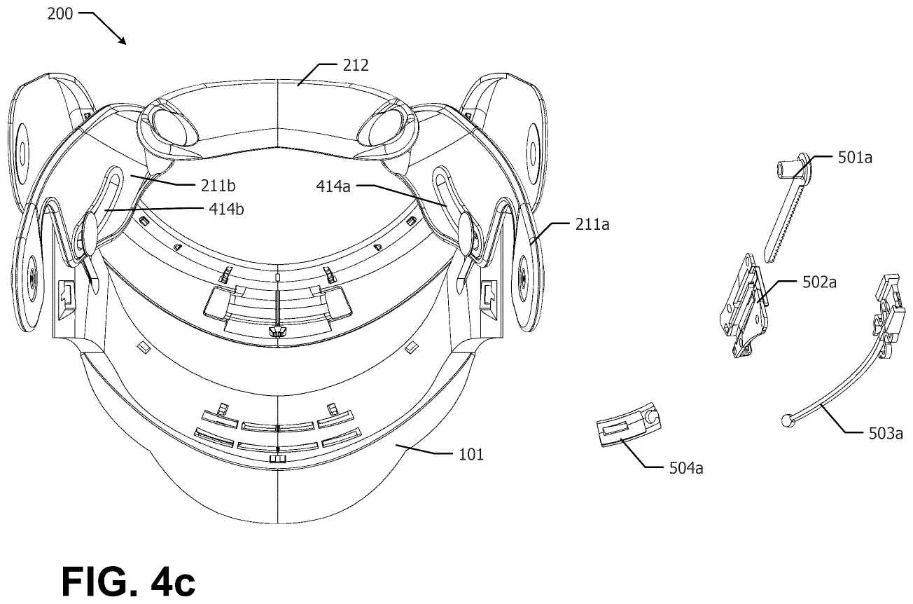

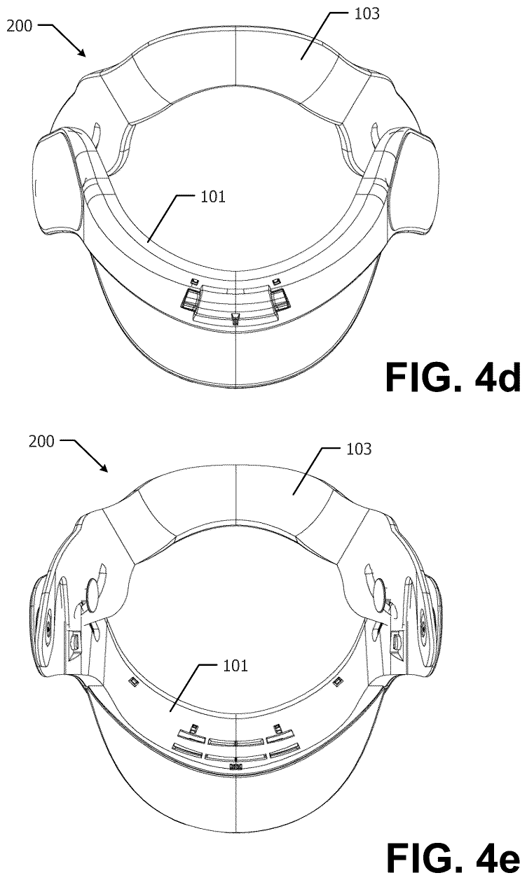

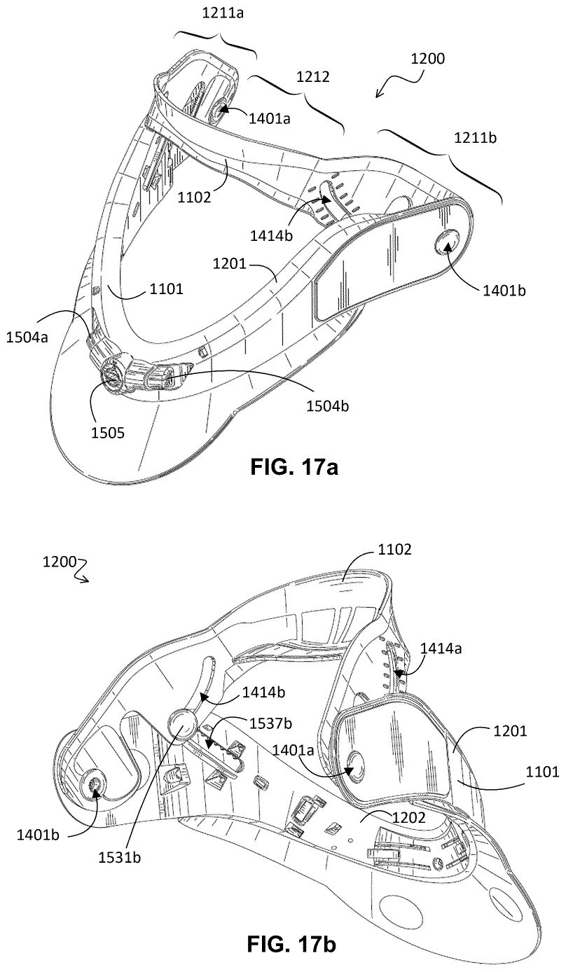

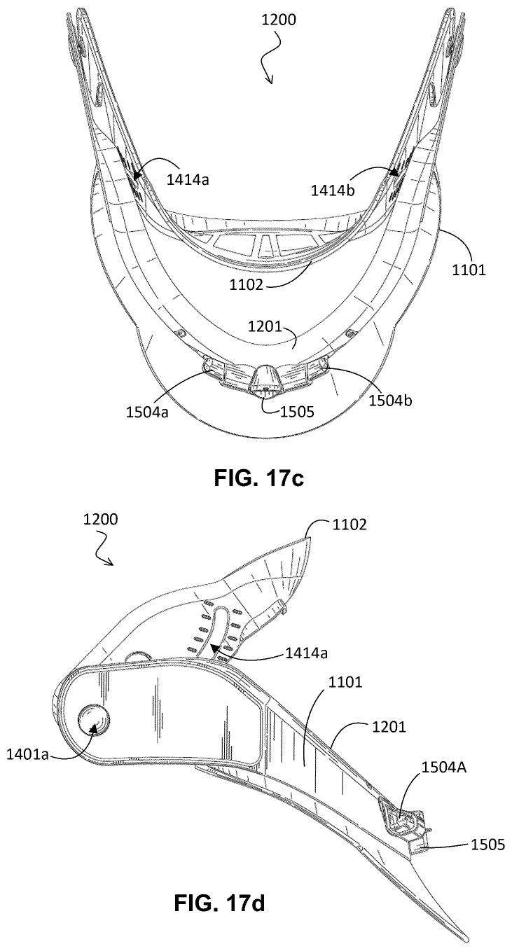

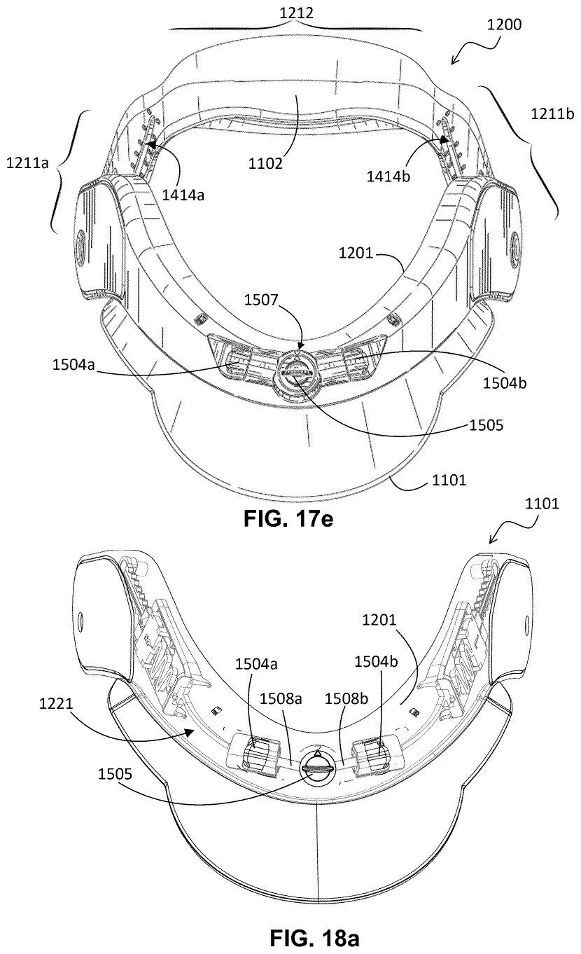

FIGS. 4a-e illustrate various views of example embodiments of an article of manufacture providing an adjustable cervical collar according to the present invention. FIG. 4a shows a front view of a front assembly 200 including the lower support arm 101 and upper chin support arm 102. The upper chin support arm 102 includes a pair of chin support arm outer components 211a-b and a chin cup 212. The chin cup 212 is coupled on each end to inner ends of each of the pair of chin support arm outer components 211a-b. In an alternate embodiment the upper chin support arm 102 may be formed as a single component in place of the pair of chin support arm outer components 211a-b and the chin cup 212 (see, e.g., 4d, 4e, & FIGS. 17a-17e at 1211a-b and 1212). FIG. 4d shows a front view of the front assembly 200 having a single piece upper chin support arm 103.

Similarly, FIG. 4e shows a rear view of the front assembly 200 having a single piece upper chin support arm 103. In addition, FIGS. 17a-17e provide various views of a front assembly 1200 having a single piece upper chin support arm 1102. As shown in FIG. 17e, the upper chin support arm 1102 can comprise a chin support section 1212, a first chin support arm outer component 1211a, a second chin support arm outer component 1211b, or a combination thereof.

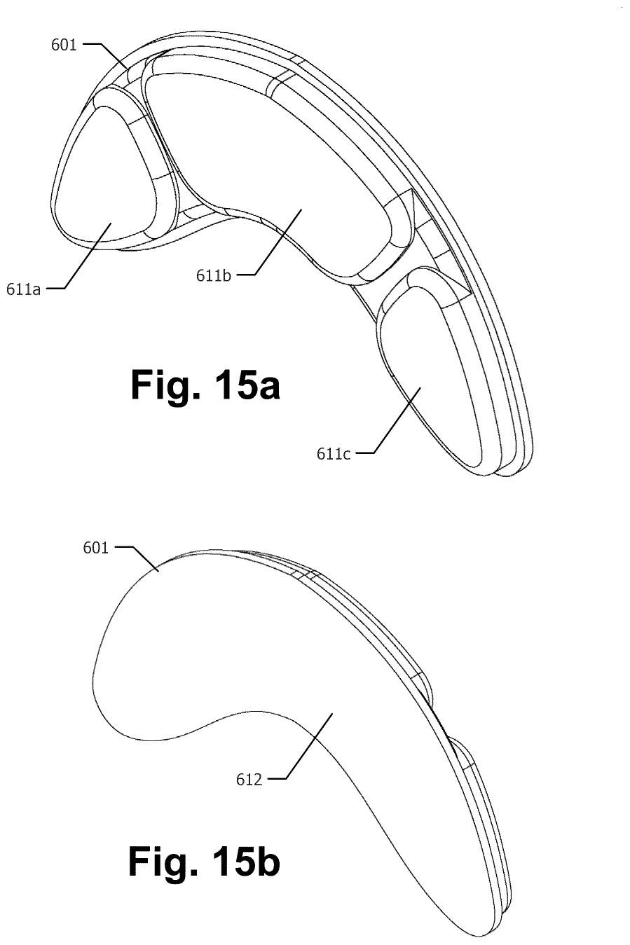

The combination of the chin cup 212, 1212 and the pair of chin support arm outer components 211a-b, 1211a-b form a rigid upper chin support arm 102, 1102 that provides support to the user's head. The chin cup 212, 1212 is configured to create a resting position for the user's chin about the center of the upper chin support arm 102, 1102. The chin cup 212, 1212 may be covered by various materials as shown in FIGS. 15a-b to provide comfort to the user when the user's chin and jaw rests upon the chin cup 212. The chin cup 212 may also include padding under any covering material.

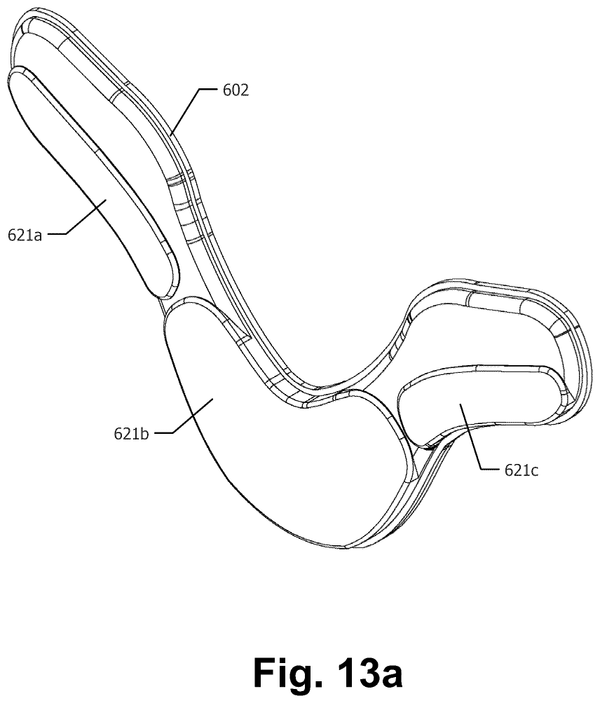

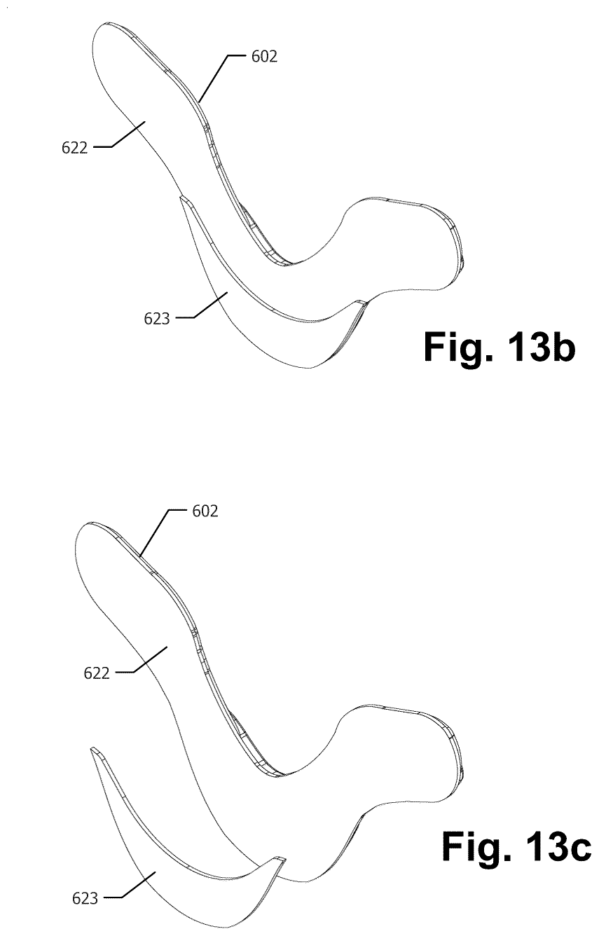

FIG. 4b shows a rear view of a front assembly 200 including the lower support arm 101 and upper chin support arm 102. The underside of the lower support arm 101 is the surface that is placed upon the user's chest when the cervical collar 100 is worn. The inner side of the lower support arm 101, in which the inner side of the lower support arm inner shell 202 is shown, is positioned about the user's neck when the cervical collar 100 is in place. Similar to the upper chin support arm 102 and the chin cup 212 described above, the lower support arm 101 may be covered by various padding materials as shown in FIGS. 13a-c to provide comfort to the user when the user's chest comes into contact with the lower support arm 101.

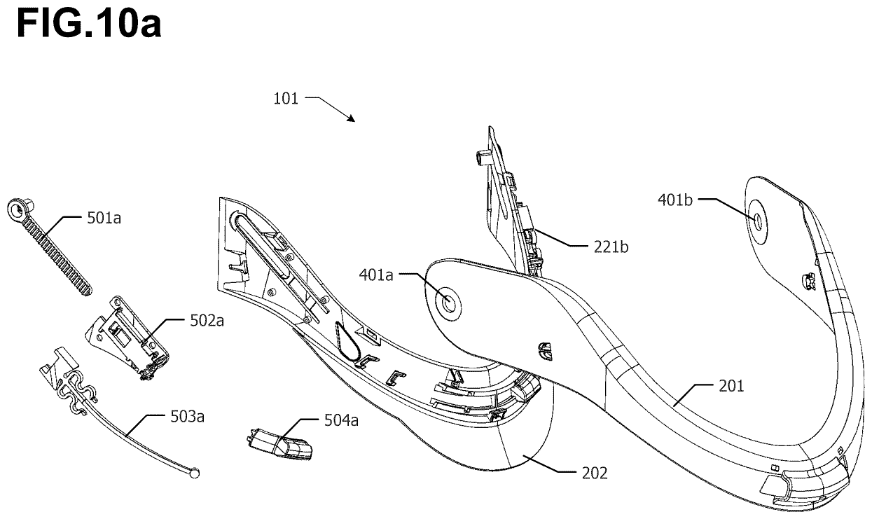

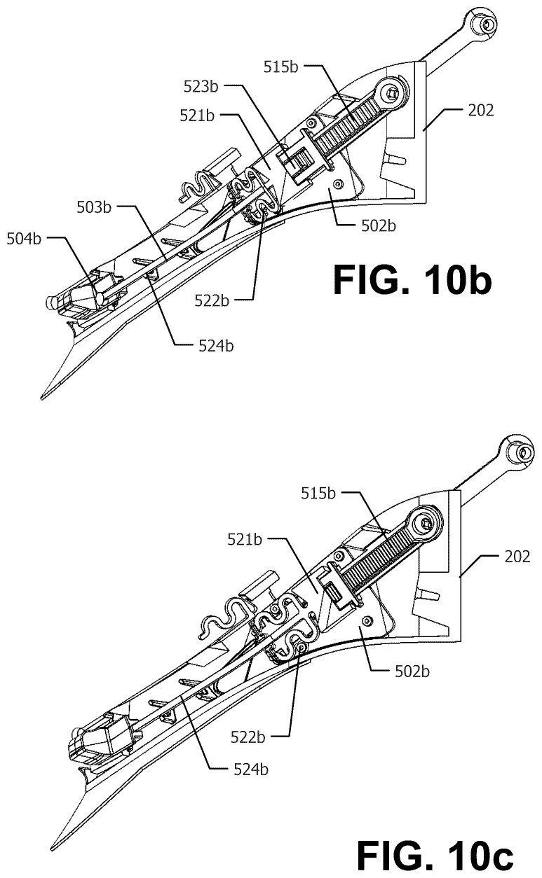

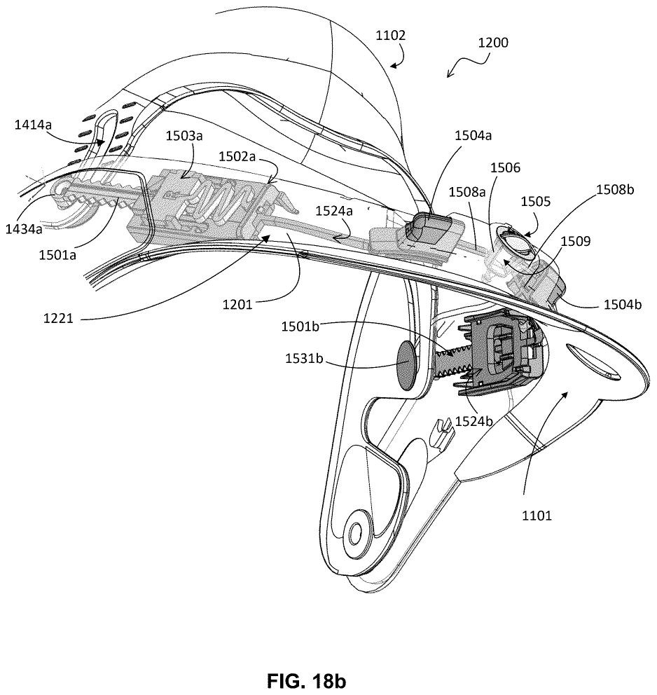

FIG. 4c shows an exploded rear view of a front assembly 200 including the lower support arm 101 and upper chin support arm 102. The components of the lower support arm locking mechanism 221a are shown adjacent to the right side of the front assembly 200. The chin cup 212 is shown in a center position of the upper chin support arm 102 between each of the chin support arm outer components 211a-b. The components of the lower support arm locking mechanism 221a include the toothed sliding height adjustment piece 501a having a plurality of teeth, the anchored locking tooth component 502a, the spring-loaded sliding lock 503a, and the locking mechanism release button 504a. The function and operation of these components within the lower support arm locking mechanism 221a-b is disclosed below in reference to FIGS. 10-12. The function and operation of similar components within the lower support arm locking mechanism 1221 of an additional embodiment is discussed below with reference to FIGS. 18-20.

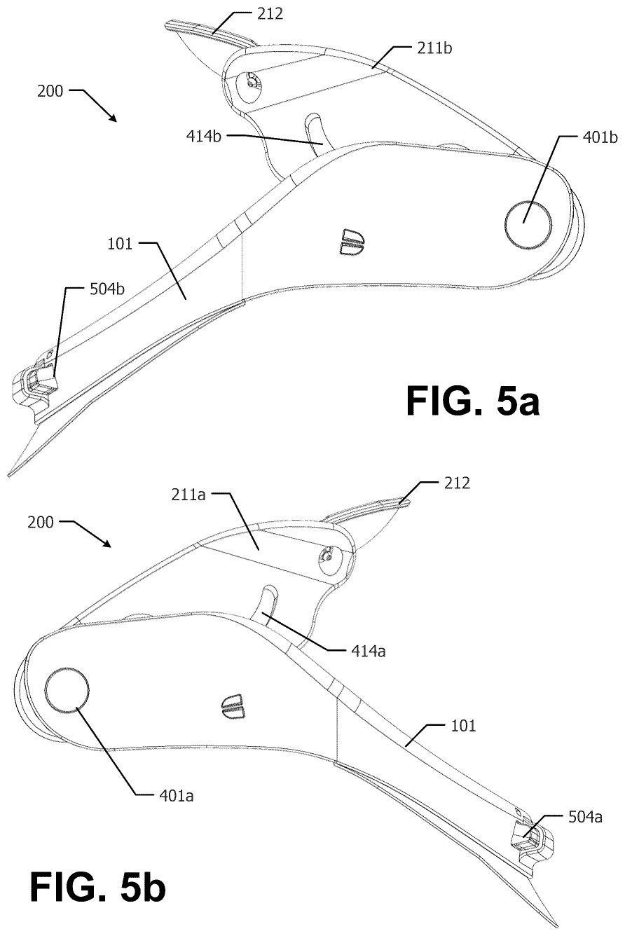

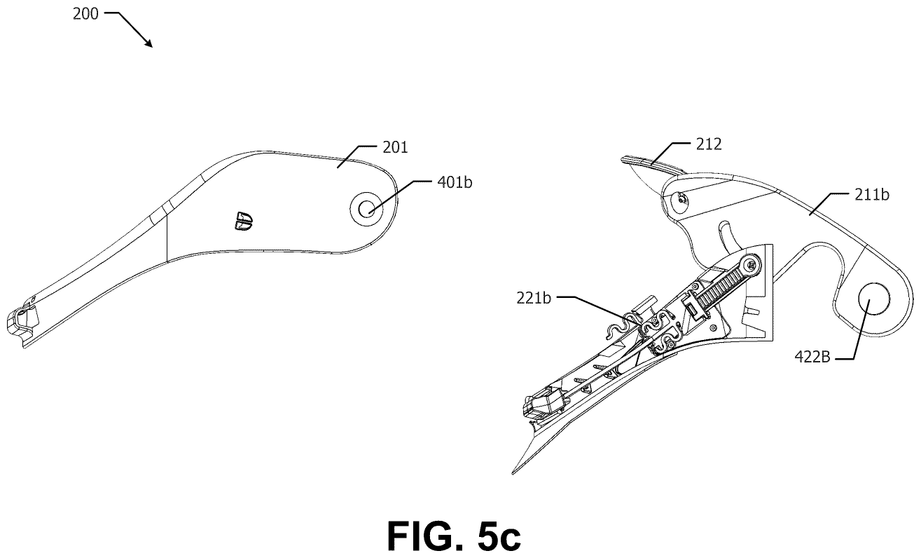

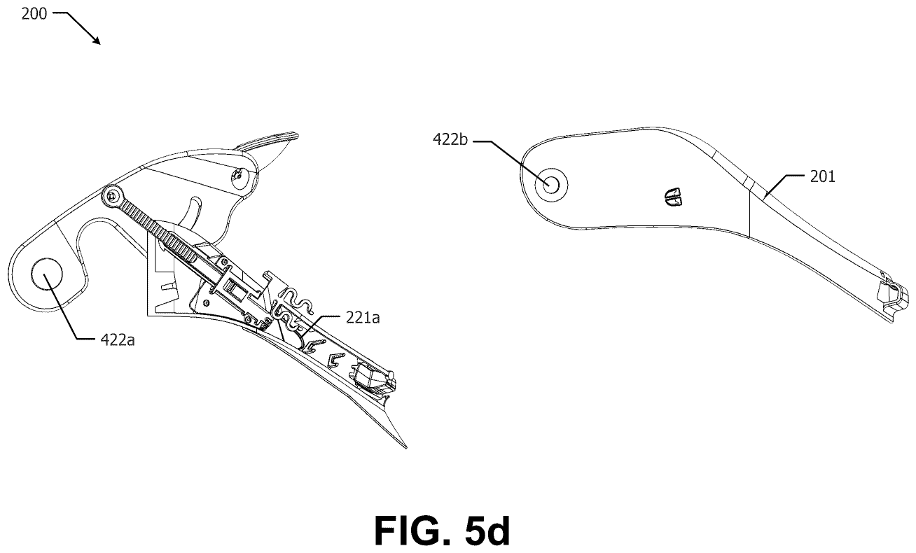

FIGS. 5a-d illustrate various side views of coupling a lower support arm and an upper chin support arm of an article of manufacture providing an adjustable cervical collar according to the present invention. FIG. 5a shows a right-side view of the lower support arm 101 pivotably coupled to the upper chin support arm 102. Similarly, FIG. 5b shows a left-side view of the lower support arm 101 pivotably coupled to the upper chin support arm 102. The lower support arm 101 has a lower support arm-upper chin support component attachment hole 401a-b through each end of the lower support arm 101, and chin support arm outer components 211a-b have upper support arm-lower support arm component attachment holes 422a-b that permit a coupling pin and receiver pair to be used to couple the lower support arm 101 and the upper chin support arm 102 together.

The lower support arm-upper chin support component attachment holes 401a-b and the upper support arm-lower support arm component attachment holes 422a-b create a pivot point about which the upper chin support arm 102 may rotate about the lower support arm 101. A pair of pivoting guide slots 414a-b is visible through the sides of the chin support arm outer components 211a-b. A pair of coupling pin and receiver pairs connect the chin support arm outer components 211a-b to the outer ends of the lower support arm 101, in which the pivoting guide slots 414a-b are configured to constrain the motion of the upper chin support arm 102 as it rotates about the pivot point between the lower support arm 101 and the upper chin support arm 102. Each of the pivoting guide slots 414a-b is curved slots through the sides of the chin support arm outer components 211a-b consistent with the rotation of the upper chin support arm 102 relative to the lower support arm 101.

The pair of locking mechanism release buttons 504a-b is visible at a center of the lower support arm 101 and connects to the lower support arm locking mechanisms 221a-b under the lower support arm outer shell 201 to lock and unlock the rotation of the upper chin support arm 102 from the lower support arm 101.

FIG. 5c shows a right-side exploded view of the lower support arm 101 pivotably coupled to the upper chin support arm 102. Similarly, FIG. 5d shows a left side exploded view of the lower support arm 101 pivotably coupled to the upper chin support arm 102. In both FIGS. 5c and 5d, the lower support arm locking mechanism 221a-b is shown attached to the lower support arm inner shell 202. The lower support arm outer shell 201 is placed over the lower support arm locking mechanism 221a-b and the lower support arm inner shell 202 and is shown to encase the lower support arm locking mechanism 221a-b within the lower support arm 101. The upper support arm-lower support arm component attachment holes 422a-b and the lower support arm-upper chin support component attachment hole 401a-b are utilized when the front assembly 200 is formed by connecting the lower support arm 101 and the upper shin support arm 102.

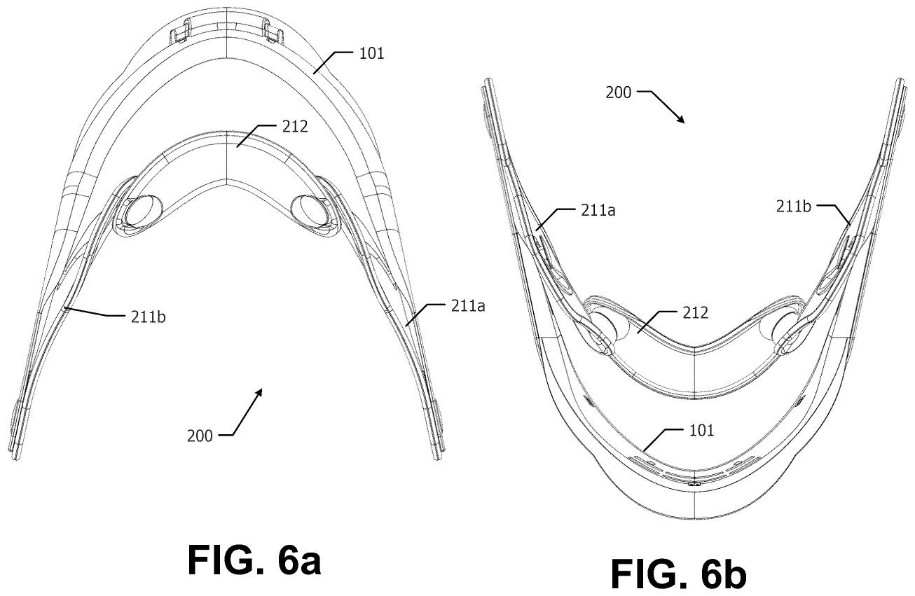

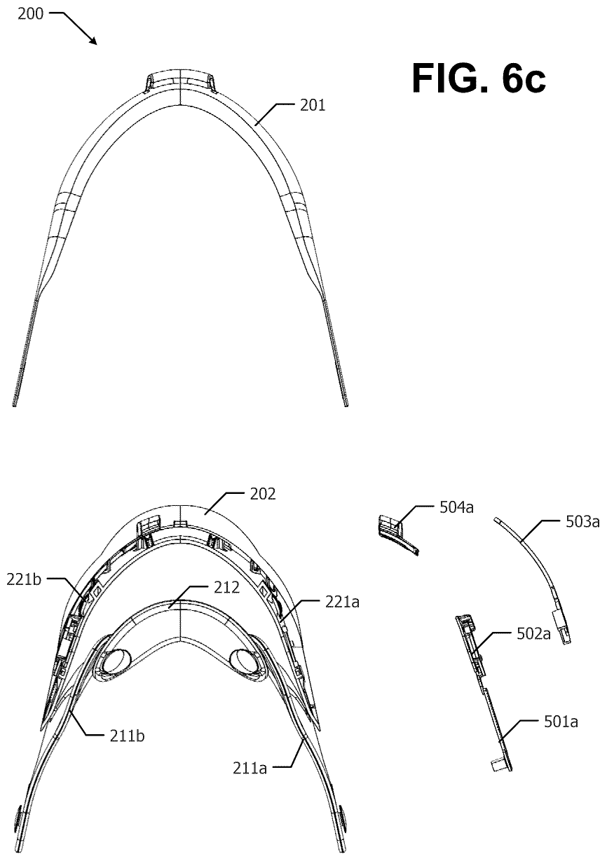

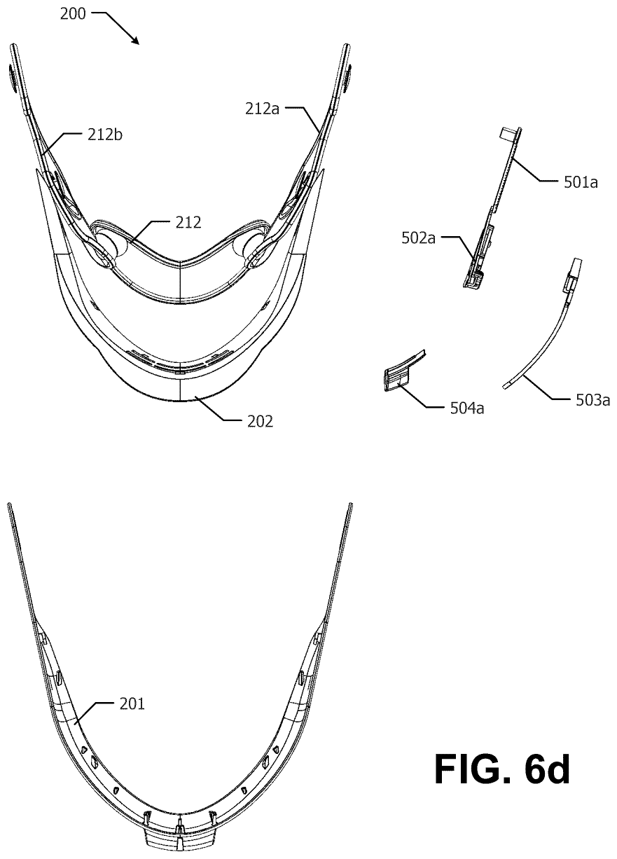

FIGS. 6a-d illustrate various top and bottom views of an article of manufacture providing an adjustable cervical collar according to the present invention. FIG. 6a shows a top view of the lower support arm 101 pivotably coupled to the upper chin support arm 102. FIG. 6b shows a bottom view of the lower support arm 101 pivotably coupled to the upper chin support arm 102. The chin cup 212 is visible within the center of the upper chin support arm 102 between the pair of chin support arm outer components 211a-b.

FIG. 6c shows an exploded top view of the lower support arm 101 pivotably coupled to the upper chin support arm 102. FIG. 6d shows an exploded bottom view of the lower support arm 101 pivotably coupled to the upper chin support arm 102. These exploded views of the front assembly 200 show the position of the pair of lower support arm locking mechanisms 221a-b. The pair of locking mechanism release buttons 504a-b is visible in the lower support arm outer shell 201 adjacent to each other within the center of the lower support arm outer shell 201.

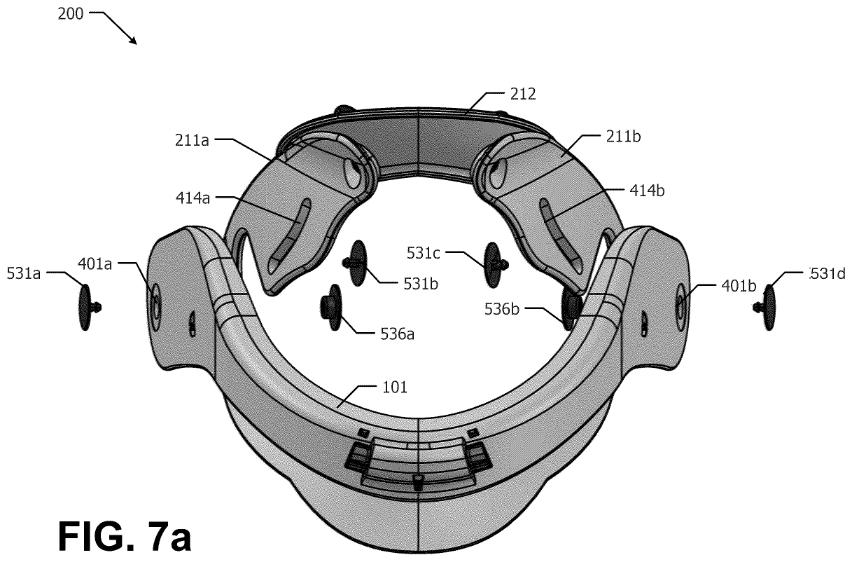

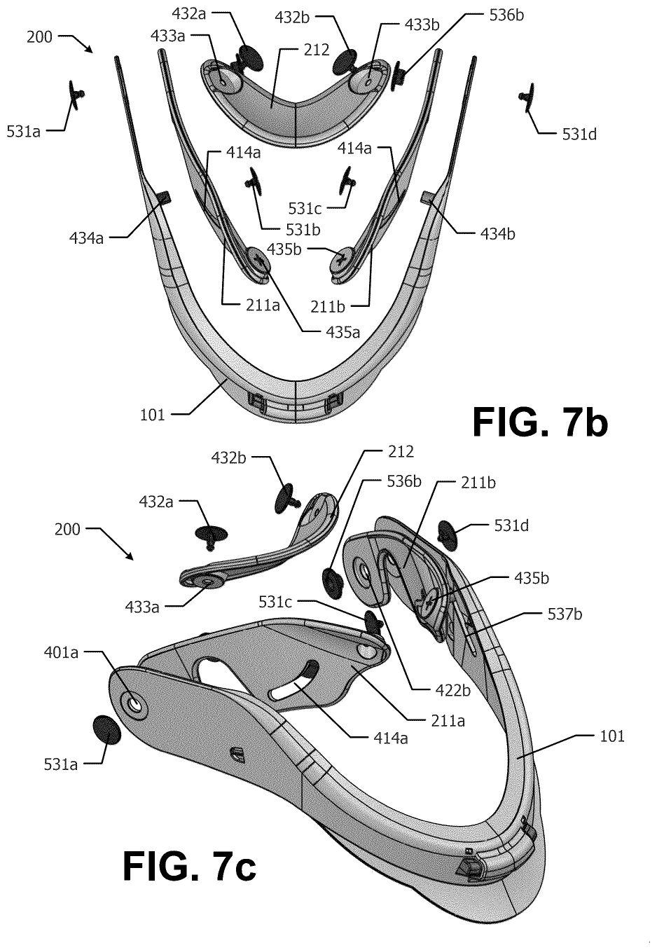

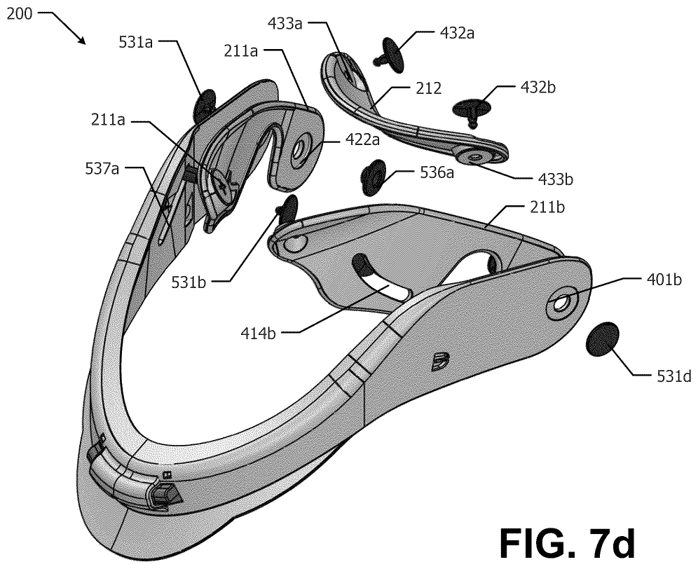

FIGS. 7a-d illustrate various views of coupling members between a lower support arm and an upper chin support arm of an article of manufacture providing an adjustable cervical collar according to the present invention. FIG. 7a shows an exploded front view of the lower support arm 101 pivotably coupled to the upper chin support arm 102 using the coupling attachment pins 531a-d and coupling attachment receivers 536a-b. The coupling attachment pins 531a, d attach to the mating coupling attachment receivers 536a-b after passing through the lower support arm-upper chin support component attachment hole 401a-b and upper support arm-lower support arm component attachment hole 422a-b. The coupling attachment pins 531a, d and coupling attachment receivers 536a-b engage each other to hold the lower support arm 101 and the chin support arm outer components 211a-b about the pivot points about the lower support arm-upper chin support component attachment holes 401a-b and upper support arm-lower support arm component attachment holes 422a-b.

The coupling attachment pins 531b, 531c couple the chin support arm outer components 211a-b to the lower support arm 101 as the coupling attachment pins 531b, c pass through the corresponding pivoting guide slots 414a-b in the chin support arm outer components 211a-b. The coupling attachment pins 531b, 531c engage within toothed sliding piece attachment pegs 434a-b as shown in figures below. The coupling attachment pins 531b, 531c create the rotational control through the pivoting guide slots 414a-b that constrain the rotation of the upper chin support arm 102 about the lower support arm 101. The toothed sliding piece attachment pegs 434a-b create height control by traveling through straight guide slots 537a-b and traverse through pivoting guide slots 414a-b. As toothed sliding piece attachment pegs 434a-b travel through both channels at the same time the angle changes between the upper chin support arm 102 and lower support arm 101.

FIG. 7b shows an exploded top view of the lower support arm 101 pivotably coupled to the upper chin support arm 102 using the toothed sliding piece attachment pegs 434a-b. In addition to the coupling attachment pins and receivers discussed above in reference to FIG. 7a, a pair of chin coupling attachment pins 432a-b and a corresponding pair of outer arm pin receivers 435a-b are shown that couple the chin support arm outer components 211a-b and the chin cup 212 together to form the upper chin support arm 102. The chin cup 212 is secured to each of the chin support arm outer components 211a-b by a pair of chin coupling attachment pins 432a-b that pass through corresponding chin cup holes 433a-b to connect to outer arm pin receivers 435a-b.

FIG. 7c shows an exploded left perspective view of the lower support arm 101 pivotably coupled to the upper chin support arm 102 using the toothed sliding piece attachment pegs 434a-b. FIG. 7d shows an exploded right perspective view of the lower support arm 101 pivotably coupled to the upper chin support arm 102. The two perspective views shown are mirror images of each other as the lower support arm 101 is coupled to the upper chin support arm 102 via the chin support arm outer components 211a-b and their corresponding attachment points.

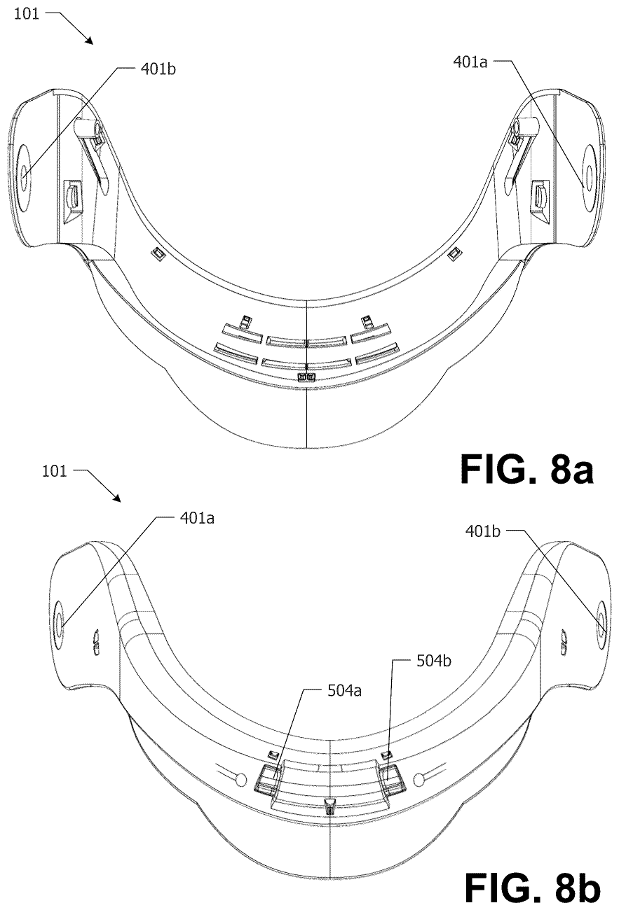

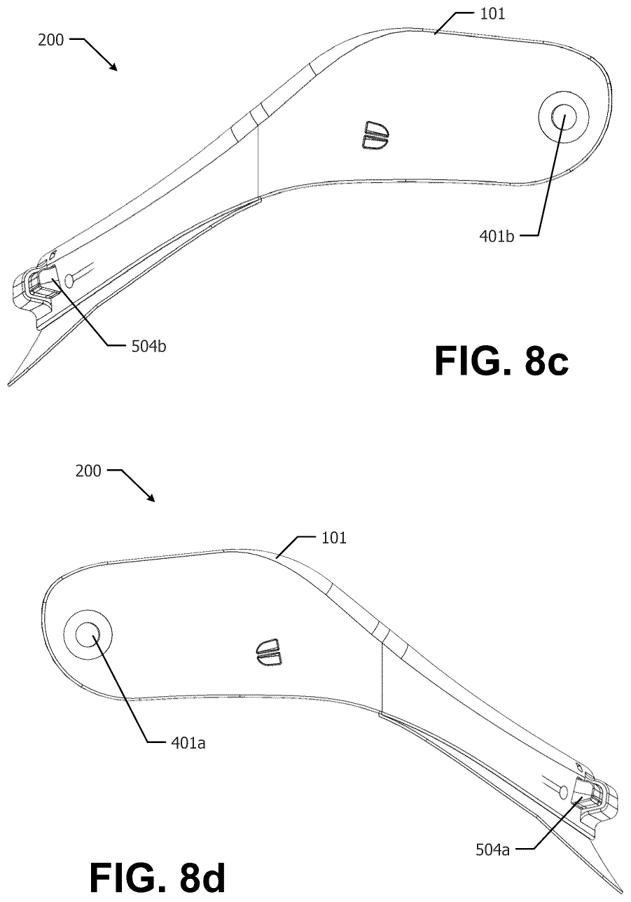

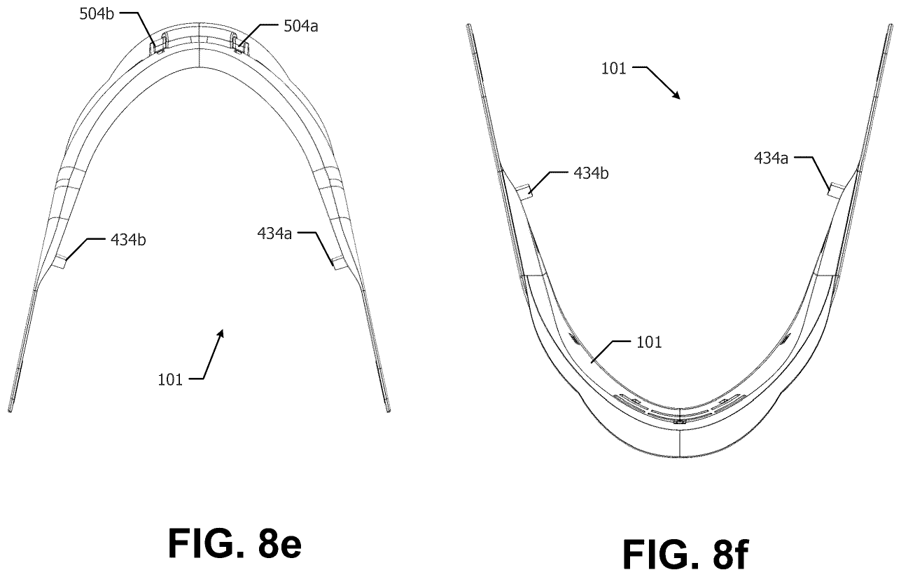

FIGS. 8a-f illustrate various views of a lower support arm of an article of manufacture providing an adjustable cervical collar according to the present invention. FIG. 8a shows a rear view of the lower support arm 101 and FIG. 8b shows a front view of the lower support arm 101. Similarly, FIG. 8c shows a right-side view of the lower support arm 101 and FIG. 8d shows a left side view of the lower support arm 101. Lastly, FIG. 8e shows a top view of the lower support arm 101 and FIG. 8f shows a bottom view of the lower support arm 101. In all of these views, the lower support arm-upper chin support component attachment holes 401a-b are shown about the outer ends of the lower support arm 101. The pair of locking mechanism release buttons 504a-b also are shown in views presenting a top side of the lower support arm 101.

FIGS. 9a-h illustrate various views of a rear support member of an article of manufacture providing an adjustable cervical collar according to the present invention. FIG. 9a shows a right perspective view of the rear support member 300 and FIG. 9b shows a left perspective view of the rear support member 300. Similarly, FIG. 9c shows a front view of the rear support member 300 and FIG. 9d shows a rear view of the rear support member 300. FIG. 9e shows a right-side view of the rear support member 300 and FIG. 9f shows a left side view of the rear support member 300. Lastly, FIG. 9g shows a top view of the rear support member 300 and FIG. 9h shows a bottom view of the rear support member 300.

In all of the above views of the rear support member 300, the rear support member 300 includes a rear support member frame component 301 and a pair of rear support member attachment loop straps 302a-b. As noted above with respect to FIGS. 3a-b, the rear support member attachment loop straps 302a-b are used to attach the rear support member frame component 301 to the front assembly 200 about a user's neck. Each of the rear support member attachment loop straps 302a-b loops through the corresponding rear support member attachment strap slots 315a-b and couples to itself. The rear support member attachment loop straps 302a-b may couple to itself after looping through the rear support member attachment strap slots 315a-b using hook and loop, adhesive, stitching, sonic welding, or similar attachment mechanism.

The rear support member frame component 301 includes a rear support member lower member 311, a pair of rear support member upper shoulder members 312a-b, and a pair of rear support member upper neck members 314a-b. The rear support member upper neck members 314a-b could be over-molded with more flexible material for more comfort. The rear support member lower member 311 is a single solid member extending downward from the center of the rear support member frame component 301 and could be an over-molded section. The rear support member lower member 311 is configured to be positioned along the upper spine of the user, typically between the shoulder blades.

Each of the rear support member upper shoulder members 312a-b extends outward from the center of the rear support member frame component 301. Both of the rear support member upper shoulder members 312a-b are configured to extend from the spine of the user both outward toward the ends of the front assembly 200 when placed about a user's neck and slightly upwards to configure the rear support member upper shoulder members 312a-b to rest across the top of the user's shoulders. Each of the rear support member upper shoulder members 312a-b is long enough to position the rear support member attachment loop straps 302a-b about the outer ends of the front assembly 200 and to hold the cervical collar 100 in place. The rear support member attachment loop straps 302a-b are configured to be attached to join the rear support member 300 to the front assembly 200 thereby creating the cervical collar 100. The rear support member attachment loop straps 302a-b may be repositioned along the adhesive hook on the front assembly 200 to adjust the tightness of the cervical collar 100 about the user's neck and head.

Each of the rear support member upper neck members 314a-b extends upward and slightly outward to create a support about a rear side of the user's neck. The rear support member upper neck members 314a-b curve inward to partially wrap around the sides of the user's neck allowing the rear support member frame component 301 to provide support to the user's head and neck while limiting the motion of the neck when the cervical collar 100 is in place.

Each of the rear support member attachment strap slots 315a-b is located within the corresponding rear support member upper shoulder members 312a-b and the rear support member attachment loop straps 302a-b are configured to loop through the rear support member attachment strap slots 315a-b and across the outer ends of the rear support member upper shoulder members 312a-b.

FIGS. 10a-c illustrate various views of a lower support arm having a pair of position locking mechanisms of an article of manufacture providing an adjustable cervical collar according to the present invention. FIG. 10a shows an exploded left perspective view of the lower support arm 101 showing the lower support arm locking mechanism 221a-b.

FIG. 10b shows a right-side view of the lower support arm locking mechanism 221a-b in an unlocked configuration. Similarly, FIG. 10c shows a right-side view of the lower support arm locking mechanism 221a-b in a locked configuration. Each of the lower support arm locking mechanisms 221a-b includes the toothed sliding height adjustment piece 501a-b, anchored locking tooth component 502a-b, the spring-loaded sliding lock 503a-b, and locking mechanism release buttons 504a-b as discussed above. The anchored locking tooth component 502a-b includes a latching tooth flexible arm portion 523a-b that engages the toothed sliding height adjustment piece 501a-b to lock the chin support arm outer components 211a-b. An upper end of each of the latching tooth flexible arm portion 523a-b is coupled to the corresponding toothed sliding height adjustment piece 501a-b, while the upper end of each of the toothed sliding height adjustment piece 501a-b is secured against the lower support arm inner shell 202 by the anchored locking tooth component 502a-b and can slide upward and downward within the anchored locking tooth component 502a-b.

Each of the spring-loaded sliding locks 503a-b has a corresponding release button coupling elongated section 524a-b release that connects the corresponding spring-loaded sliding lock 503a-b to the locking mechanism release buttons 504a-b. The locking mechanism release buttons 504a-b extend outward through the lower support arm outer shell 201 permitting the user to depress the locking mechanism release buttons 504a-b to unlock the lower support arm locking mechanism 221a-b.

When a user depresses one or both locking mechanism release buttons 504a-b, the locking mechanism release buttons 504a-b move toward the center of the lower support arm 101. The spring-loaded sliding lock 503a-b is pulled downward as the locking mechanism release button 504a-b is depressed causing the spring-loaded sliding lock 503a-b to slide downward. A spring engagement mechanism 522a-b is coupled to a top end of the spring-loaded sliding lock 503a-b and is configured to be positioned within the anchored locking tooth component 502a-b to disengage the tooth engagement body 521a-b from the latching tooth flexible arm portion 523a-b. When the tooth engagement body 521a-b slides downward, it disengages the latching tooth flexible arm portion 523a-b permitting the latching tooth flexible arm portion 523a-b to freely lift off of the toothed sliding height adjustment piece 501a-b to cause the chin support arm outer component 211a-b to unlock. When the lower support arm locking mechanism 221a-b is unlocked, the toothed sliding height adjustment piece 501a-b may slide up and down within the lower support arm locking mechanism 221a-b as the upper chin support arm 102 rotates with respect to the lower support arm 101.

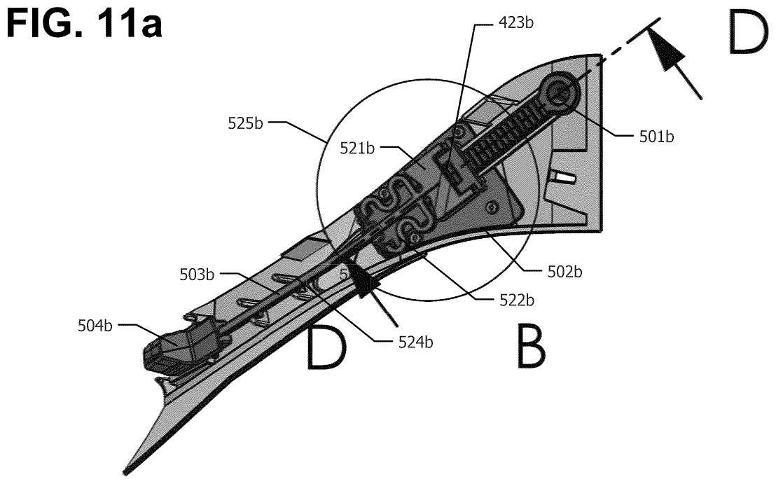

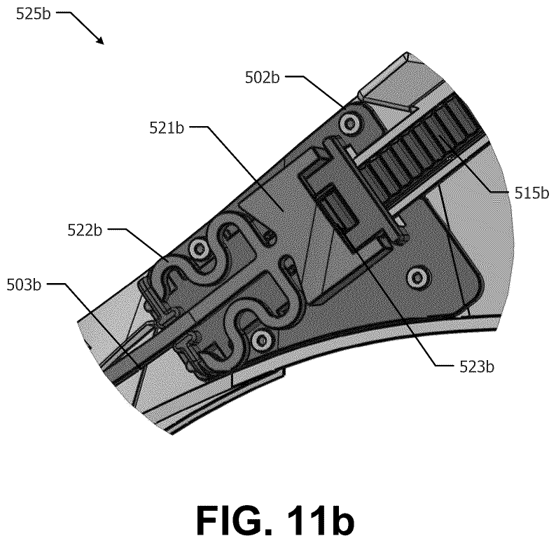

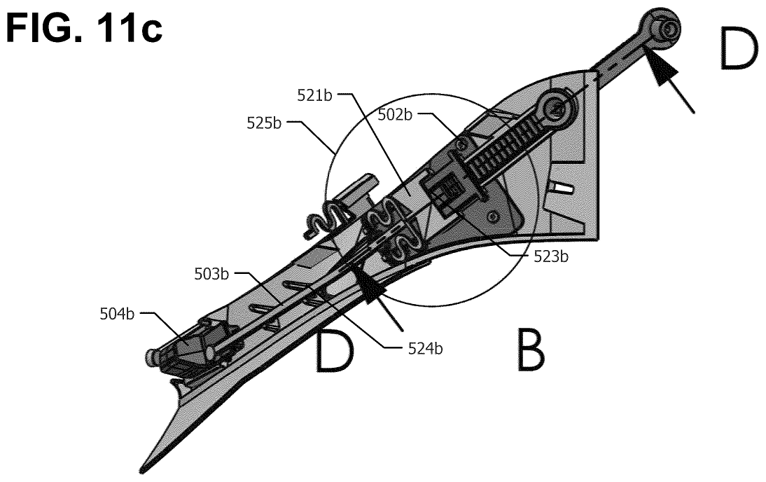

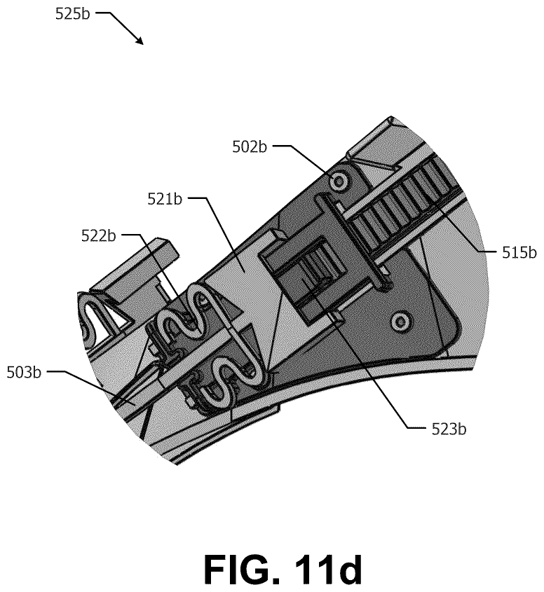

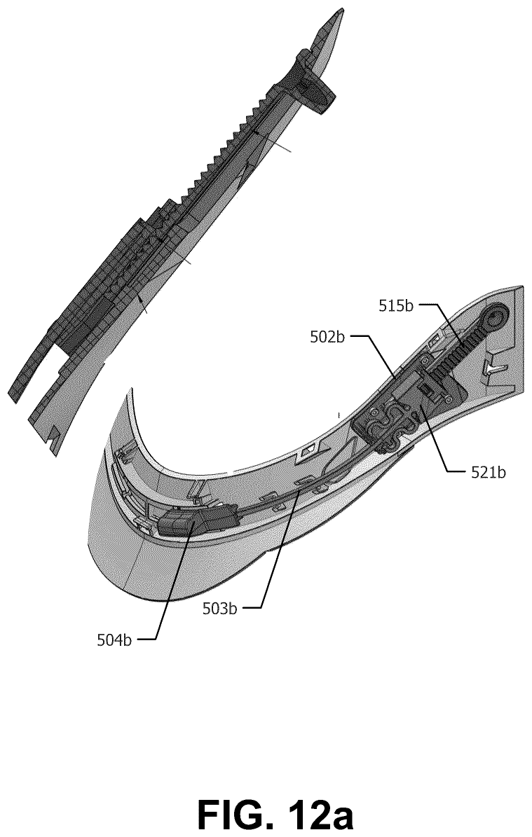

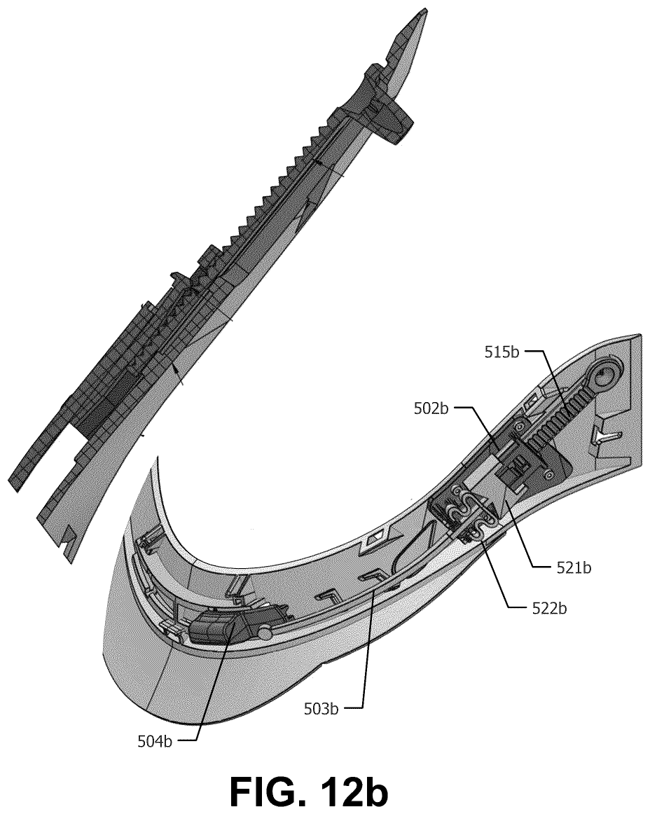

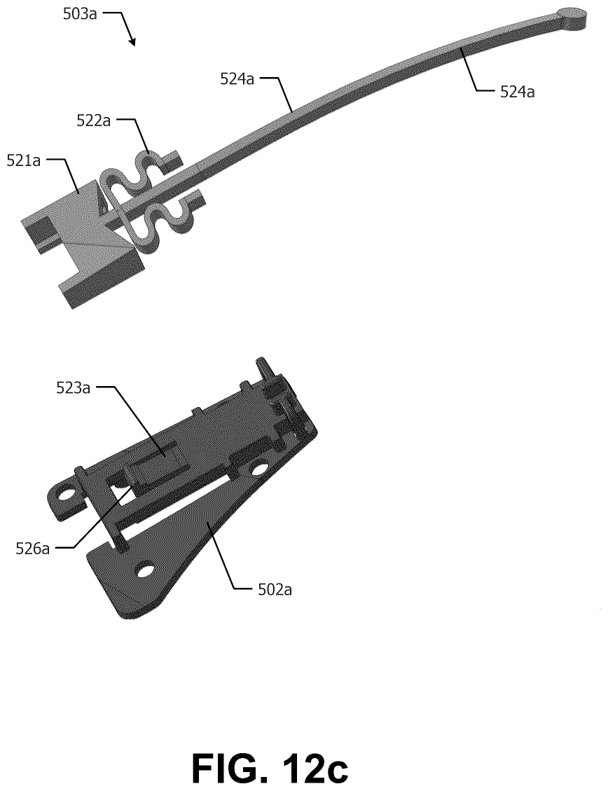

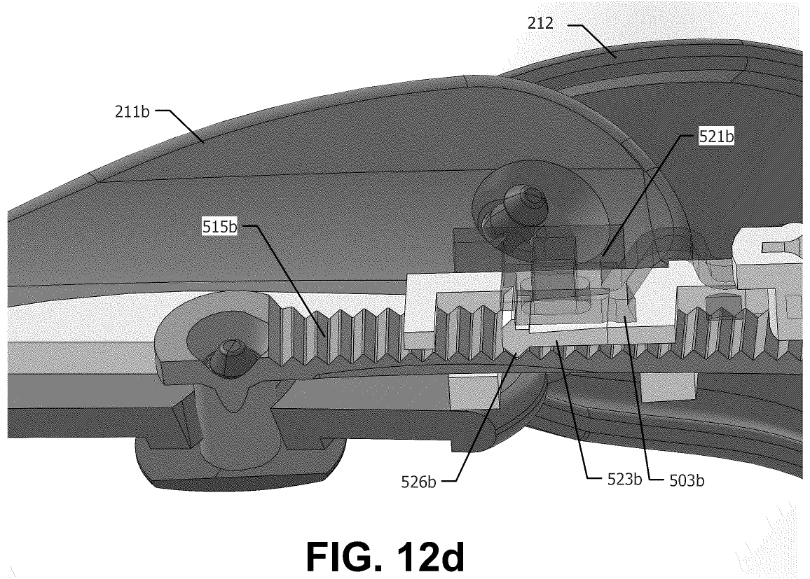

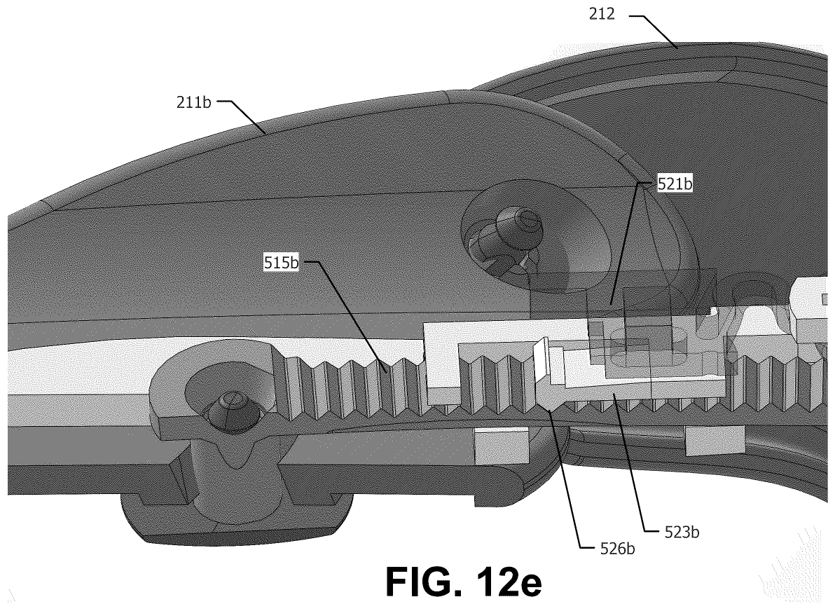

When the user releases the locking mechanism release buttons 504a-b, the spring-loaded sliding lock 503a-b is pulled upward by the spring engagement mechanism 522a-b allowing the tooth engagement body 521a-b to slide to reengage the latching tooth flexible arm portion 523a-b forcing the arm downward to reengage the toothed sliding height adjustment piece 501a-b to relock the lower support arm locking mechanism 221a-b. The operation of the latching mechanism in the lower support arm locking mechanism 221a-b is disclosed in detail below in reference to FIGS. 11a-f and FIGS. 12a-e.

FIG. 11a-d illustrate various views of components of each of the pair of position locking mechanisms of an article of manufacture providing an adjustable cervical collar according to the present invention. FIG. 11a shows a right side view of the lower support arm locking mechanism 221a-b in a locked position. A corresponding detailed right side view 525b is shown in FIG. 11b of the lower support arm locking mechanism 221a in a locked position. Similarly, FIG. 11c shows a right side view of the lower support arm locking mechanism 221a in an unlocked position. A corresponding detailed right side view 525b is shown in FIG. 11d of the lower support arm locking mechanism 221a in an unlocked position.

In FIG. 11a-b, the spring engagement mechanism 522a-b are shown in an uncompressed configuration which creates interference between the spring engagement mechanism 522a-b and the anchored locking tooth component 502a-b. The interference between the spring engagement mechanism 522a-b and the anchored locking tooth component 502a-b prevents the tooth engagement body 521a-b from disengaging with the latching tooth flexible arm portion 523a-b, pushing the arm downward to engage the teeth in the toothed sliding height adjustment piece 501a-b, preventing the toothed sliding height adjustment piece 501a-b from sliding upward or downward within the anchored locking tooth component 502a-b. When the toothed sliding height adjustment piece 501a-b is not constrained, it may slide upward and downward within straight guide slot 537a-b to permit the upper chin support arm 102 to rotate about the lower support arm 101.

FIGS. 12a-d illustrate open and closed configurations of each of the pair of position locking mechanisms of an article of manufacture providing an adjustable cervical collar according to the present invention. FIG. 12a shows a left side view of the locking mechanism latching teeth 515a-b that engage the lower support arm locking mechanism 221a-b when the locking mechanism is configured in a locked position. Similarly, FIG. 12b shows a left side view of the locking mechanism latching teeth 515a-b that engages the lower support arm locking mechanism 221a-b when the locking mechanism is configured in an unlocked position. The operation of the lower support arm locking mechanism 221a-b in these figures is described in detail above with reference to FIGS. 10-11.

FIG. 12c shows components of anchored locking tooth component 502a-b and spring-loaded sliding lock 503a-b. The spring-loaded sliding lock 503a-b includes the release button coupling elongated section 524a-b, a spring engagement mechanism 522a-b, and a tooth engagement body 521a-b. The anchored locking tooth component 502a-b includes the latching tooth flexible arm portion 523a-b coupled at an inner end of the arm to a top surface of the anchored locking tooth component 502a-b. Each of the latching tooth flexible arm portion 523a-b further includes an engagement tooth 526a-b at an outer end of the latching tooth flexible arm portion 523a-b that engages the locking mechanism latching teeth 515a-b of the toothed sliding height adjustment piece 501a-b. The spring-loaded sliding lock 503a-b slides across the top surface of anchored locking tooth component 502a-b when the spring engagement mechanism 522a-b are uncompressed as shown in FIG. 12d. When the spring engagement mechanism 522a-b are pulled into a compressed configuration when the locking mechanism release buttons 504a-b are depressed by the user, the spring-loaded sliding lock 503a-b slide in the direction of the locking mechanism release buttons 504a-b to disengage the latching tooth flexible arm portion 523a-b allowing the arm to rise free from the locking mechanism latching teeth 515a-b of the toothed sliding height adjustment piece 501a-b as shown in FIG. 12e.

FIGS. 13a-c illustrate multiple views of a lower support arm padding 602 used with the lower support arm 101 of an article of manufacture providing an adjustable cervical collar according to the present invention. FIG. 13a shows a front perspective view of the lower support arm padding 602 having a plurality of raised padding sections 621a-c. FIG. 13b shows a rear perspective view of the lower support arm padding 602 showing a rear surface 622 and a lower pocket outer item 623 coupled to the rear surface 622 of the lower support arm padding 602. FIG. 13c shows an exploded rear perspective view of the lower support arm padding 602 showing the lower pocket outer item 623. The lower pocket outer item 623 may be utilized to wrap around corresponding components of the lower support arm 101 to hold the lower support arm padding 602 in place when the adjustable cervical collar 100 is in use.

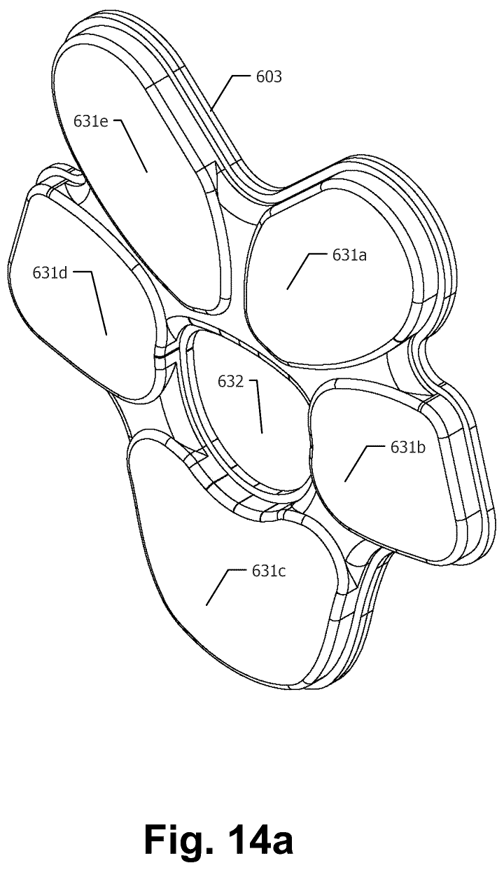

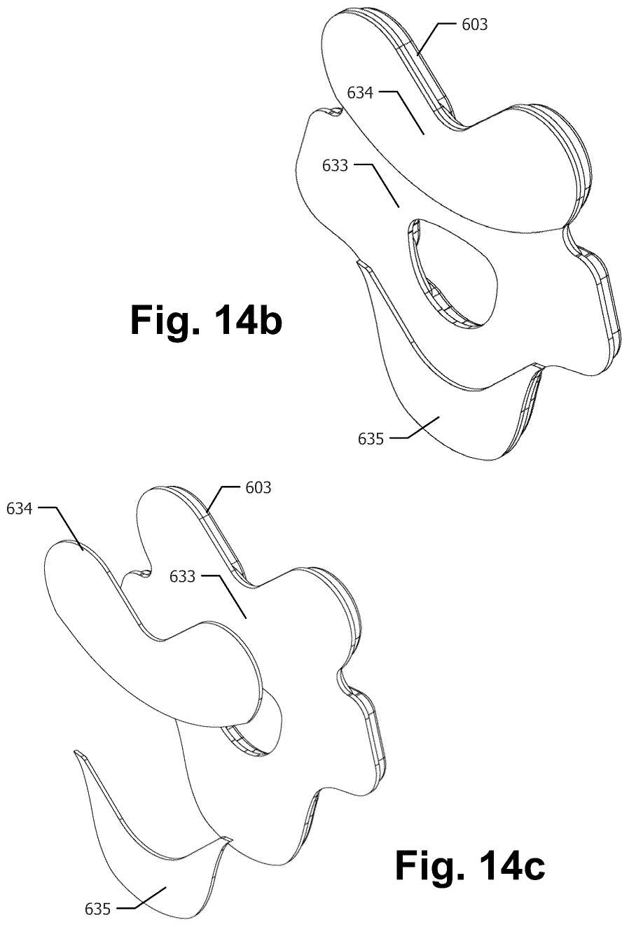

FIGS. 14a-c illustrate multiple views of a rear support member padding 603 used with the rear support member 300 of an article of manufacture providing an adjustable cervical collar according to the present invention. FIG. 14a shows a front perspective view of the rear support member padding 603 having a plurality of raised padding sections 631a-e and a center hole 632. FIG. 14b shows a rear perspective view of the rear support member padding 603 showing a rear surface 633, a top pocket outer item 634, and a lower pocket outer item 635 coupled to a rear surface 633 of the rear support member padding 603. FIG. 14c shows an exploded rear perspective view of the rear support member padding 603 showing the rear surface 633, the top pocket outer item 634, and a lower pocket outer item 635. The top pocket outer item 634 and the lower pocket outer item 635 may be utilized to wrap around corresponding components of the rear support member 300 to hold the rear support member padding 603 in place when the adjustable cervical collar 100 is in use.

FIGS. 15a-b illustrate multiple views of an upper chin support arm padding 601 used with the upper chin support arm 102 of an article of manufacture providing an adjustable cervical collar according to the present invention. FIG. 15a shows a front perspective view of the upper chin support arm padding 602 having a plurality of raised padded sections 611a-c. FIG. 15b shows a rear perspective view of upper chin support arm padding 601 having a rear surface 612. Attachment mechanisms including hook and loop devices, adhesive, and similar devices may be utilized to hold the upper chin support arm padding 601 in place when the adjustable cervical collar 100 is in use.

With respect to the padding items including the lower support arm padding 602, upper chin support arm padding 601, and the rear support member padding 603, the padding may be constructed from padding material over-molded into a desired shape, cut and shaved into a desired shape, or shaped into a desired configuration using similar construction methods. The padding items 601-603, each item may be covered using fabric or similar materials that are laminated, sewn, or otherwise attached to the padding to provide a desired outer surface for contacting the user when the adjustable cervical collar 100 is worn.

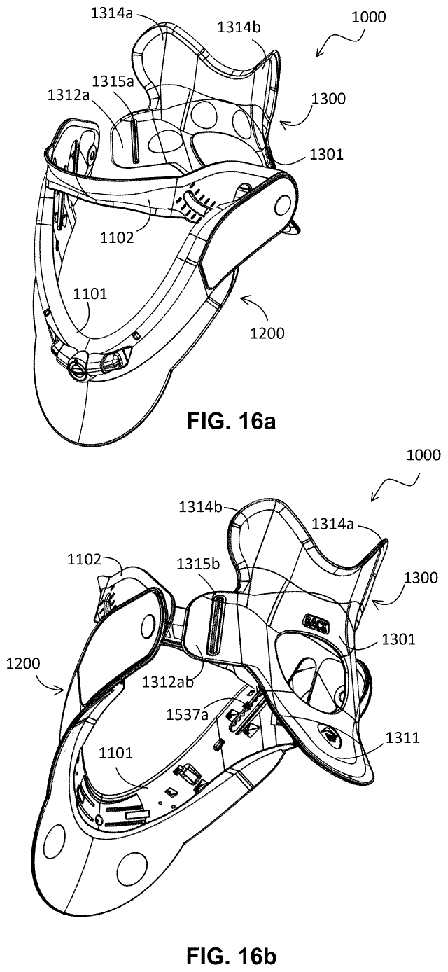

FIGS. 16a and 16b illustrate a top perspective view and a bottom perspective view, respectively, of an adjustable cervical collar 1000 under another embodiment. The adjustable cervical collar 1000 comprises a front assembly 1200, a rear assembly (also referred to herein as a “rear support member”) 1300, or a combination thereof. In embodiments, the front assembly 1200 may be provided separately from the rear assembly 1300 or vice versa.

As can be seen the cervical collar 1000 under the embodiments of FIGS. 16a and 16b can comprise features or elements that are similar in form or function to other embodiments disclosed herein. Further, it should be understood that any of the features or elements of the various embodiments disclosed herein can be interchangeable with like-named features from another embodiment unless the context dictates otherwise. Namely, any of the features or elements of the embodiments of FIGS. 16a-b can be incorporated into any of the alternate embodiments disclosed herein. Likewise, it should be understood that any features or elements disclosed with respect to FIGS. 16a-16b can possess a function that is similar or identical to any like-named features disclosed with respect to alternate embodiments.

As shown in FIGS. 16a and 16b, the rear support member 1300 includes a rear support member frame component 1301 with a rear support member attachment strap slot 1315a-b disposed at opposite ends thereof. In embodiments, the rear support member attachment strap slots 1315a-b can be configured to receive rear support member attachment straps (such, but not limited to, as those seen, e.g., at 302a-b of FIGS. 3a-b), which can be used to attach the rear support member frame component 1301 to the front assembly 1200 about a user's neck when in use.

The rear support member frame component 1301 can include a rear support member lower member 1311, a pair of rear support member upper shoulder members 1312a-b, and a pair of rear support member upper neck members 1314a-b. In embodiments, the rear support member upper shoulder members 1312a-b have a length that is sufficient to position the rear support member attachment loop straps (shown at 302a-b of FIGS. 3a-b) about the outer ends of the front assembly 1200 and to hold the cervical collar 1000 in place. The rear support member attachment loop straps (shown at 302a-b of FIGS. 3a-b) can be configured to join the rear support member 1300 to the front assembly 1200 thereby creating the cervical collar 1000. Although a hook and loop mechanism is shown as one example of joining the front assembly 1200 with the rear support member 1300, one of skill in the art would understand that alternate means can be applied to reversibly join the front assembly 1200 with the rear support member 1300. Non-limiting, exemplary means of such attachment mechanisms include buttons, friction snaps, clips, buckles, zippers, and the like.

As shown in the embodiment of FIGS. 16a-b, the front assembly 1200 can comprise a lower support arm 1101 coupled to a pivotable upper chin support arm 1102. The general operation of the embodiments of FIG. 16a-b occurs in a similar manner to that described in alternate embodiments disclosed herein. Briefly, the position of the upper chin support arm 1102 and lower support arm 1101 with respect to one another can be configured a user-defined configuration and reversibly locked in such a user-defined configuration. With the lower support arm 1101 resting upon the user's chest, the user can rest his or her chin on top of the upper chin support arm 1102, allowing the front assembly 200 to support the weight of the user's head.

FIGS. 17a-e provide various views of the front assembly 1200 of FIGS. 16a-b. The lower support arm 1101 is seen at the bottom of the front assembly 1200 and the upper chin support arm 1102 is shown at an upper portion of the front assembly 1200. In this embodiment, the upper chin support arm 1102 is a single piece that comprises a chin support section (also referred to herein as a “chin cup”) 1212, a first chin support arm outer component 1211a, a second chin support arm outer component 1211b, or a combination thereof. As with alternate embodiments disclosed herein, the upper chin support arm 1102 and the lower support arm 1101 are pivotally connected via a pivot point 1401a-b, and height and rotational control of the cervical collar 1000 can be achieved via cooperative interaction between guide slots 1414a-b, 1537a-b, attachment pegs 1434a-b, coupling attachment pins 1531a-b, and the locking mechanism (seen at 1221 of FIGS. 18, 19, and 20) in a similar manner as discussed above with reference to the FIG. 7 embodiments.

Briefly, a pair of pivoting guide slots 1414a-b can be seen in FIGS. 17a-e, which extend through the sides of the chin support arm outer components 1211a-b of the upper chin support arm 1102. Each of the pivoting guide slots 1414a-b can be curved to permit controlled rotation of the upper chin support arm 1102 relative to the lower support arm 1101. A pair of straight guide slots (seen 1537a of FIGS. 16b and 1537b of 17b) can extend through the inner shell 1202 of the lower support arm 1101, wherein each of the straight guide slots 1537a-b is configured to permit an attachment peg (seen at 1434a-b of FIGS. 18b, 19, 20a-c and 434a-b of FIGS. 7b and 8f) to extend therethrough.

Consistent with the description of FIG. 7, movement of the attachment pegs (seen at 1434a-b of FIGS. 18b, 19, 20a-c and 434a-b of FIGS. 7b and 8f) in conjunction with coupling attachment pins (seen at 1531a-b and 531b, 531c) within the pivoting guide slots 1414a-b and the straight guide slots 1537a-b permits rotational control such that a user can create a customizable, user-defined configuration of the lower support arm 1101 relative to the upper chin support arm 1102. Thus, in operation, the attachment pegs 1434a-b contribute to height control by traveling first through straight guide slots (seen at 1537a-b of FIGS. 16b and 17b, respectively) in the lower support arm 1101 and then through the pivoting guide slots 1414a-b of the upper chin support arm 1102. As the attachment pegs 1434a-b travel through both channels 1414a-b, 1537a-b at the same time, the angle changes between the upper chin support arm 1102 and lower support arm 1101.

In the embodiment of FIG. 17a, the lower support arm 1101 comprises an outer shell 1201 that is coupled to an inner shell (seen at 1202 of FIG. 17 b). A pair of locking mechanism release buttons 1504a-b can be seen partially extending out of the lower support arm outer shell 1201. In operation, one or more locking mechanism release buttons 1504a-b connect to the lower support arm locking mechanisms (seen at 1221 of FIGS. 18, 19, and 20 under the lower support arm outer shell 1201 to lock and unlock the rotation of the upper chin support arm 1102 from the lower support arm 1101. As shown in the present embodiment, the lower support arm 1101 can comprises a safety lock 1505 or other means for preventing accidental or unintentional depression of one or both of the locking mechanism release buttons 1504 a-b. The safety lock 1505 can permit the adjustable cervical collar 1000 to alternate between a locked or “secured configuration,” wherein depression of one or more locking mechanism release buttons 1504a-b is inhibited or prevented, and an unlocked or “unsecured configuration,” wherein depression of the one or more locking mechanism release buttons 1504a-b (and subsequent adjustment of the cervical collar 1000) is neither inhibited nor prevented.

By way of example, the safety lock 1505 can comprise a locking knob 1505. In embodiments and as shown in FIG. 17, the locking knob 1505 is centrally disposed between the two locking mechanism release buttons 1504a-b. As shown in FIG. 17e, the lower support arm 1101 can comprise a lock indicator 1507 configured to alert the user to whether the adjustable cervical collar 1000 is in a secured or an unsecured configuration. FIG. 17e shows the locking knob 1505 with the lock indicators 1507 aligned to signify that the cervical collar 1000 is in the “secured configuration.” By contrast, when the lock indicator 1507 in the embodiment of FIG. 17e is not aligned (not pictured), the cervical collar 1000 assumes the “unsecured configuration.” In alternative embodiments, alignment of the lock indicator 1507 signifies that the cervical collar 1000 is in an unsecured configuration and misalignment of the lock indicators signifies that the cervical collar 1000 is in a secured configuration. It should be understood that the present disclosure also includes alternate types and configurations of lock indicators that would be known to one of skill in the art.

FIGS. 18a-b provide a front and bottom perspective views, respectively, of the lower support arm 1101, with the outer shell 1201 shown in phantom and the lower shell (seen at 1202 of FIG. 17) removed to reveal the configuration of the locking knob 1505, the one or more locking mechanism release buttons 1504a-b, and the lower support arm locking mechanisms 1221 therewithin. As shown in this exemplary embodiment, the locking knob 1505 can be rotatably disposed within the lower support arm 1101 and comprise at least one blocking wall 1506 and a passthrough 1509. Each of the one or more locking mechanism release buttons 1504a-b can also comprise a medially extending locking extension 1508a-b. As most clearly shown in FIG. 18b, when in the secured configuration, the one or more blocking walls 1506 are aligned in a manner that prevents the one or more locking extensions 1508a-b from moving in the medial direction (toward the center of the lower support arm 1101). Thus, when in the secured configuration, a user cannot depress the one or more locking mechanism release buttons 1504a-b, preventing adjustment of the cervical collar 1000. To achieve an unlocked or “unsecured configuration,” the locking knob can be twisted or rotated such that the blocking walls 1506 are no longer aligned in a manner that prevents the one or more locking extensions 1508a-b from moving in the medial direction. When so arranged, the cervical collar 1000 assumes the unsecured configuration, to permit depression of one or more locking mechanism release buttons 1504a-b such that locking extensions 1508a-b move medially into the passthrough 1509 of the locking knob, and the locking mechanism 1221 can be unlocked to for adjustment of the cervical collar 1000.

The lower support arm locking mechanism 1221 can be seen underneath the outer shell 1201, which is shown in phantom in FIGS. 18a-b.

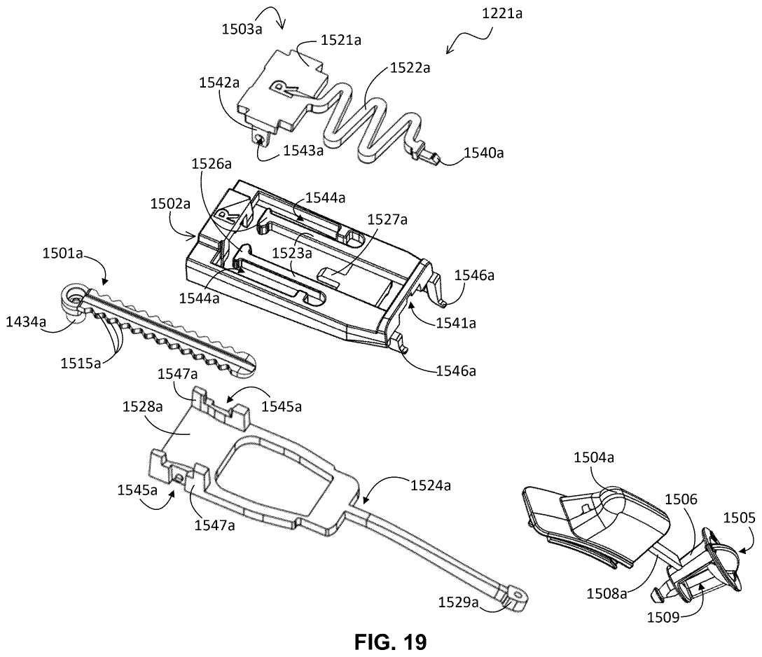

FIG. 19 illustrates an exploded, top perspective view of the right-side lower support arm locking mechanism 1221a of FIG. 18a. The right-side lower support arm locking mechanism is shown isolated from the lower support arm. It should be understood that such isolation is made solely for ease of reference, in operation, the locking mechanism 1221 will be disposed within the lower support arm 101, 1101 of the various embodiments disclosed herein to permit locking, unlocking, and adjustment of the user-defined cervical collar configuration. For clarity, only elements from the right side of the locking mechanism 1221a are shown in FIG. 19. However, it should be understood that (as disclosed in additional figures herein), the left side of the locking mechanism can comprise identical or similar elements that are disclosed and discussed with reference to FIG. 19. Thus, any disclosure of a feature or element in FIG. 19 that is numerically identified with the letter “a” (indicating the right-side version of the feature, e.g., 1503a) also discloses the same element on the left side of the embodiment, which is denoted with the letter “b” (e.g., 1503b) in additional figures disclosed herein.

As can be seen from the FIG. 19 embodiment, the locking mechanism 1221a can comprise at least one toothed sliding height adjustment piece 1501a, at least one anchored locking tooth component 1502a, at least one spring-loaded sliding lock 1503a, at least one elongated connector 1524a, at least one locking mechanism release button 1504a, or any combination thereof. The term “elongated connector” is used interchangeably with the term “elongated section” in the present disclosure.

The toothed sliding height adjustment piece 1501a can comprise an attachment peg 1434a on one end thereof. The toothed sliding height adjustment piece 1501 can also comprise a plurality of locking mechanism latching teeth 1515a, which, in the FIG. 19 embodiment, are disposed on opposite lateral sides of the toothed sliding height adjustment piece 1501a. Thus, each toothed sliding height adjustment piece 1501a can comprise two locking surfaces (a first locking surface and a second locking surface) that are opposite one another, wherein each locking surface comprises a plurality of latching teeth 1515a. In alternate embodiments, such as that disclosed in FIGS. 10-12, the toothed sliding height adjustment piece 501 comprises only a single locking surface with a plurality of latching teeth 515. Cervical collar embodiments that include a toothed sliding adjustment piece 1501a with at least two locking surfaces can be utilized to provide a stronger locking mechanism as compared to embodiments that include a sliding adjustment piece 501a with only one locking surface.

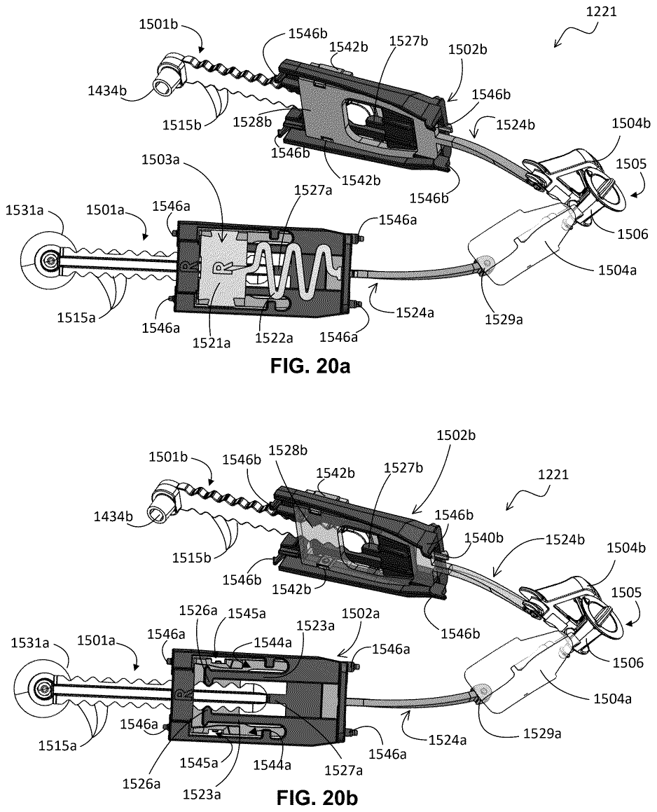

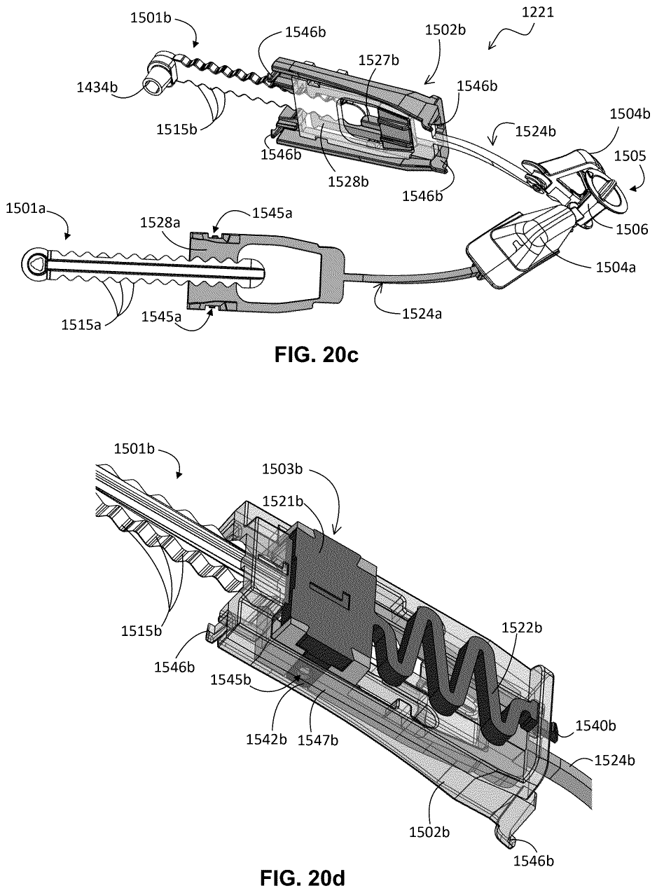

As shown in FIG. 19, the spring-loaded sliding lock 1503a can comprise an arm engagement body 1521a and a spring mechanism 1522a. In the pictured embodiment, the arm engagement body 1521a is disposed at the back half of the spring-loaded sliding lock 1503a and the spring mechanism 1522a is disposed at the front half of the spring-loaded sliding lock 1503a. An anchor prong 1540a can be seen extending from the frontmost portion of the spring-loaded sliding lock 1503a. In embodiments, the anchor prong 1540a is configured to connect at least a portion of the spring-loaded sliding lock 1503a to a prong receiving channel 1541a of the anchored locking tooth component 1502a. As shown, the arm engagement body 1521a can comprise at least one arm engagement projection 1542a, which extends away from the arm engagement body 1521a and toward the anchored locking tooth component 1502a. The arm engagement projection 1542a can comprise a connector pin receiving hole 1543a, which can be dimensioned to receive a connection pin in the projection receiving channel 1545a of the elongated connector 1524a. In embodiments, the arm engagement projection 1542a is dimensioned to extend through the projection sliding guide 1544a of the anchored locking tooth component 1502a and into the projection receiving channel 1545a of the elongated connector 1524a such that the connector pin receiving hole 1543a is aligned with a laterally projecting pin extending from the elongated connector 1524a and into the projection receiving channel 1545a. As more clearly seen in FIG. 20, the spring-loaded sliding lock 1503a can comprise at least two oppositely disposed arm engagement projections 1542a.

The anchored locking tooth component 1502a can in include at least one latching tooth flexible arm portion 1523a that further includes an engagement tooth 1526a extending in the medial direction at one end thereof. The engagement tooth 1526 can comprise a profile that is configured to fit with the grooves between the latching teeth 1515a of the toothed sliding height adjustment piece 1501a. As shown in FIG. 19, the anchored locking tooth component 1502a can comprise two latching tooth flexible arm portions 1523a, wherein, a first latching flexible arm portion 1523a and its associated medially projecting engagement tooth 1526 are configured to interact with the plurality of latching teeth 1515a on a first locking surfaces of the sliding adjustment piece 1501a and a second latching flexible arm portion 1523a and its associated medially projecting engagement tooth 1526 are configured to interact with the plurality of latching teeth 1515a on a second locking surface of the sliding adjustment piece 1501a.

A projection sliding guide 1544a can be seen lateral to each of the latching flexible arm portions 1523a of the anchored locking tooth component 1502a. In embodiments, the projection sliding guide 1544a is dimensioned to permit at least a portion of the arm engagement projections 1542a of the spring-loaded sliding lock 1503a to pass therethrough. The anchored locking tooth component 1502a can further include an angled abutment member 1527a that is configured to contact the forward-most portion of the toothed sliding height adjustment piece 1501a. In operation the angled abutment member 1527a can direct the forward-most portion of the toothed sliding height adjustment piece to move inward (toward the middle of the cervical collar 1000) as the height adjustment piece 1501a moves in the forward direction during adjustment of the cervical collar 1000 as described in more detail below.

The anchored locking tooth component 1502a can comprise a plurality of anchoring clips 1546 that are configured to attach or anchor the anchored locking tooth component 1502a to the inner shell 1202 of the lower support arm 1101. When so attached, the anchored locking tooth component is immobilized to the lower support arm 1101. As more clearly seen in FIG. 20a, anchored locking tooth component 1502a can comprise at least four anchoring clips 1546, with an anchoring clip 1546a disposed at each corner of the anchored locking tooth component 1502a.

As seen in FIG. 19, the elongated connector 1524a can comprise a forward extending arm that terminates at the frontmost portion in a connector flange 1529a. In embodiments, the connector flange 1529a is configured to attach the elongated connector 1524a to the locking mechanism release button 1504a. As can be seen, when so connected, inward depression of the locking mechanism release button 1504a causes the elongated connector 1524a to be pulled forward, which, as discussed in more detail below, ultimately results in unlocking of the locking mechanism 1221. Toward the back end, the elongated connector 1524a comprises a connector body 1528a. In embodiments, the connector body 1528a comprises at least one laterally disposed connector wall 1547a, which can project away from the connector body 1528a in the direction of the spring-loaded sliding lock 1503a. As can be seen, the embodiment of FIG. 19 comprises two laterally disposed connector walls 1547a, which are disposed on opposite sides of the connector body 1528a. The at least one connector wall 1547a can include a projection receiving channel 1545a that is configured to receive and hold at least a portion of the arm engagement projection 1542a of the spring-loaded sliding lock 1503a therein. As shown in FIG. 19, the connector wall 1547a can comprise a laterally projecting extension or pin that extends into the projection receiving channel, which is dimensioned to fit within the connector pin receiving hole 1543a of the arm engagement projection 1542a to ensure a strong connection between the elongated connector 1524a and the spring-loaded sliding lock 1503a. By way of such connection between the elongated connector 1524a and the spring-loaded sliding lock 1503a, the arm engagement body 1521a moves forward with the elongated connector 1524a during depression of the locking mechanism release button 1504a.

FIGS. 20a-c provide right perspective views of the locking mechanism 1221 utilized in the exemplary embodiments of FIGS. 16-19. Collectively, FIGS. 20a-d provide detailed views of the locking mechanism 1221 to reveal the connections between each element utilized therein. The complete locking mechanism 1221 or portions thereof are shown isolated from the outer shell and inner shell of the lower support arm 1101. As with FIG. 19, it should be understood that such isolation is made solely for ease of reference, in operation, the locking mechanism 1221 will be disposed within the lower support arm 101, 1101 of the various embodiments disclosed herein to permit locking, unlocking, and adjustment of the user-defined cervical collar 100, 1000 configuration. Briefly, in operation, at least of portion of the locking mechanism 1221 is coupled to the lower support arm inner shell (seen at 1202 of FIG. 17) and the entire locking mechanism 1221 is encased within the lower support arm outer shell (seen at 1201 of FIGS. 17 and 18). It should also be noted that, while each of the pictured embodiments of FIGS. 20a-20d show the locking mechanism in a locked position, when read in view of the present specification, one of skill in the art would understand how manipulation of the locking mechanism 1221 as described herein could result in unlocking of the cervical collar to permit adjustment of the user-defined configuration.

FIG. 20a provides a top, side perspective view of the locking mechanism 1221 of the FIG. 18a embodiment. The locking mechanism 1221 is shown isolated from the lower support arm (seen at 1101 of FIGS. 18a-b), and the right locking mechanism release button 1504a is shown in phantom.

FIG. 20b provides a top, side perspective partial view of the locking mechanism 1221 of the FIG. 18a embodiment. In this view, the partial locking mechanism is shown isolated from the lower support arm (seen at 1101 of FIGS. 18a-b), and the right spring-loaded sliding lock (seen at 1503a of FIG. 20a) is removed from the locking mechanism 1221 to provide an unobstructed view of the right anchored locking tooth component 1502a. In addition, the right locking mechanism release button 1504a and left elongated connector 1524b are shown in phantom.

FIG. 20c provides a top, side perspective partial view of the locking mechanism 1221 of the FIG. 18a embodiment. In this view, the partial locking mechanism 1221 is shown isolated from the lower support arm (seen at 1101 of FIGS. 18a-b). The right spring-loaded sliding lock (seen at seen at 1503a of FIG. 20a) and the right anchored locking tooth component (seen at 1502a of FIG. 20b) are removed from the locking mechanism 1221 to provide an unobstructed view of the right toothed sliding height adjustment piece 1501a and a clearer view of the right elongated connector 1524a. The left spring-loaded sliding lock (seen at 1503a of FIG. 20d) is also removed. In addition, the left elongated connector 1524b is shown in phantom to reveal the mechanism by which the anchored locking tooth component 1502b interacts with the toothed sliding height adjustment piece 1501b such that the toothed sliding height adjustment piece 1501b is reversibly locked therein.

FIG. 20d provides a bottom, side perspective partial view of a portion of the left locking mechanism 1221b of the FIG. 18a embodiment. In this view, the partial locking mechanism 1221b is shown isolated from the lower support arm (seen at 1101 of FIGS. 18a-b). The left anchored locking tooth component 1502b is shown in phantom.

As can be seen, the locking mechanism 1221 in the embodiment of FIGS. 16-20 is configured such that the spring-loaded sliding lock 1503a-b is immobilized at the front end (via the anchor prong 1540a-b) but is free to move forward and backward at the at the back end (via the arm engagement body's 1521a-b connection to the elongated connector 1524a-b) such that the spring portion 1522a-b expands and compresses therebetween. Briefly, in operation of one embodiment, the spring-loaded sliding lock 1503a-b is anchored at the front end by the anchor prong 1540a-b to the anchored locking tooth component 1502a-b, and the anchored locking tooth component 1502a-b is attached to the inner shell (seen at 1202 of FIG. 17) of the lower support arm 1101 by a plurality of anchoring clips 1546a-b. Thus, the anchored locking tooth component 1502a-b is anchored to the lower support arm 1101 and cannot move with respect thereto. Similarly, the front end of the spring-loaded sliding lock 1503a-b is immobilized by virtue of its connection to the anchored locking tooth component 1502a-b via the anchor prong 1540a-b. By contrast, at the opposite end (front end) of the spring-loaded sliding lock 1503a-b, the arm engagement body 1521a-b is connected via the arm engagement projection 1542a-b to the elongated connector 1524a-b. Thus, the arm engagement body 1521a-b of the spring-loaded sliding lock 1503a-b can move forward and backward with the elongated connector 1524a-b.

As can be seen from the above, in operation of the cervical collar 1000, the arm engagement projection 1542a-b of the spring-loaded sliding lock 1503a-b is disposed within the projection sliding guide 1544a-b of the anchored locking tooth component 1502a-b such that, when in an unlocked state, the arm engagement projection 1542a-b can slide forward (toward the front of the cervical collar 1000) and backward (toward the back of the cervical collar 1000) within the projection sliding guide 1544a-b. When in a locked position, the spring mechanism 1522a-b pushes the arm engagement body 1521a-b toward the back of the locking mechanism 1221. The reward pressure on the sliding lock 1503a-b when the spring 1522a-b in such a decompressed state positions the arm engagement body 1521a-b such that the arm engagement projection 1542a-b is disposed toward the back of the projection sliding guide 1544a-b and lateral to the engagement teeth 1526a-b of the flexible arms 1523a-b of the anchored locking tooth component 1502a-b. When so disposed, the arm engagement projection 1542a-b prevents lateral flexion of the portion of each flexible arm 1523a-b corresponding to the arm's associated engagement tooth 1526a-b. Thus, when in the locked position, each engagement tooth 1526a cannot be laterally displaced from the grooves between the latching teeth 1515a-b of the toothed sliding height adjustment piece 1501a-b. As a result, when the locking mechanism 1221 is in a locked position, the toothed sliding height adjustment piece 1501a-b is restricted from moving within the anchored locking tooth component 1502a-b.

By contrast, when the locking mechanism release buttons 1504a-b are depressed, the elongated connector 1524a-b is pulled in the forward and medial direction, which moves the arm engagement body 1521a-b of the spring-loaded sliding lock in the forward direction and compresses spring mechanism 1522a-b. As the arm engagement body 1521a-b moves forward, the arm engagement projection 1542a-b slides toward the front of the locking mechanism 1221 within the projection sliding guide 1544a-b of the anchored locking tooth component 1502a-b. As the arm engagement projection 1542a-b slides toward the front of the locking mechanism 1221, the arm engagement projection 1542a-b moves away from the portion of each flexible arm 1523a-b corresponding to the arm's associated engagement tooth 1526a-b. Thus, when the locking mechanism release buttons 1504a-b are depressed, the arm engagement projection 1542a-b no longer prevents lateral flexion of the portion of the flexible arms 1523a-b associated with the engagement tooth 1526a-b, and the engagement tooth 1526a-b can be laterally displaced from the grooves between the latching teeth 1515a-b of the toothed sliding height adjustment piece 1501a-b. As a result, when the locking mechanism 1221 is in an unlocked position, the toothed sliding height adjustment piece 1501a-b can move forward or backward within the anchored locking tooth component 1502a-b to permit the associated attachment pegs 1434a-b to move within the pivoting guide slots 1414a-b and straight guide slots 1537a-b and enable adjustment of the cervical collar 1000.

Finally, after a desired user-defined configuration is achieved, the locking mechanism release buttons 1504a-b can be released. Upon such release, compression of the spring mechanism 1522a-b of the spring-loaded sliding lock 1503a-b is also released, and the arm engagement body 1521a-b moves backward and returns to the locked position. As discussed above, in the locked position, the arm engagement projections 1542a-b are disposed lateral to the engagement teeth 1526a-b portion of the flexible arms 1523a-b of the anchored locking tooth component 1502a-b, preventing the engagement teeth 1526a from being laterally displaced from the grooves between the latching teeth 1515a-b of the toothed sliding height adjustment piece 1501a-b, thereby locking the cervical collar 1000 in the desired configuration.

Even though particular combinations of features are recited in the present application, these combinations are not intended to limit the disclosure of the invention. In fact, many of these features may be combined in ways not specifically recited in this application. In other words, any of the features mentioned in this application may be included in this new invention in any combination or combinations to allow the functionality required for the desired operations.

No element, act, or instruction used in the present application should be construed as critical or essential to the invention unless explicitly described as such.

Further, the phrase “based on” is intended to mean “based, at least in part, on” unless explicitly stated otherwise.

What is claimed is:

1. An adjustable cervical collar comprising:
a lower support arm having a pair of opposing ends;
a locking mechanism coupled to the lower support arm and having a locked and unlocked configuration;
a pivotable upper chin support arm having opposing ends and coupled to the lower support arm at the opposing ends;
wherein the lower support arm and the upper chin support arm rotate about a connecting pivot point when the lower support arm locking mechanism is in an unlocked configuration; and
the lower support arm locking mechanism constrains the pivoting positioning of the lower support arm relative to the pivotable upper chin support arm in the locked configuration.

2. The adjustable cervical collar according to claim 1, wherein the lower support arm locking mechanism comprises:
a toothed sliding height adjustment piece comprising at least one locking surface, wherein the locking surface comprises a plurality of teeth;
an anchored locking tooth component;
a spring-loaded sliding lock;
an elongated section; and
a locking mechanism release button.

3. The adjustable cervical collar according to claim 2, wherein the anchored locking tooth component comprises:
a latching tooth flexible arm; and
an engagement tooth at an end of the latching tooth flexible arm.

4. The adjustable cervical collar according to claim 3, wherein the spring-loaded sliding lock comprises:
an arm engagement body; and
a spring mechanism.

5. The adjustable cervical collar according to claim 4, wherein the at least one locking surface comprises a plurality of grooves disposed between the plurality of teeth, each of the grooves comprising a shape that is complementary to that of the engagement tooth such that, when in the locked configuration, at least one of the grooves receives the engagement tooth therein.

6. The adjustable cervical collar according to claim 5, wherein
the spring-loaded sliding lock comprises an anchor prong that is disposed at one end of the spring-loaded sliding lock and the arm engagement body is disposed at an end of the spring-loaded siding lock that is opposite to that of the anchor prong such that the spring mechanism is disposed between the anchor prong and the arm engagement body.

7. The adjustable cervical collar according to claim 6, wherein
the spring mechanism is attached to the anchored locking tooth component via the anchor prong;
the locking mechanism release button is attached to the elongated section; and
the elongated section is attached to the arm engagement body of the spring-loaded sliding lock.

8. The adjustable cervical collar according to claim 2, wherein the lower support arm comprises:
a lower support arm outer shell; and
a lower support arm inner shell, the locking mechanism is coupled to the lower support arm inner shell.

9. The adjustable cervical collar according to claim 8, wherein at least a portion of the locking mechanism release button extends through a portion of the lower support arm outer shell such that a portion of the locking mechanism release button is accessible.

10. The adjustable cervical collar according to claim 2, further comprising a rear support member attachable from the lower support arm, the rear support member comprising:
a rear support member frame component and
a pair of upper shoulder members, wherein the rear support member is attachable to the lower support arm via the upper shoulder members.

11. The adjustable cervical collar according to claim 10, wherein the rear support member frame component comprises:
a pair of rear support member attachment strap slots for accepting corresponding rear support member attachment straps;
a rear support member lower member; and
a pair of rear support member upper neck members.

12. The adjustable cervical collar according to claim 1, further comprises:
a lower support arm padding coupled to the lower support arm using one or more first lower pocket outer item;
a rear support member padding coupled to the rear support member using a top pocket outer item and a second lower pocket outer item; and
an upper chin support member padding coupled to the upper chin support arm.

13. The adjustable cervical collar according to claim 12, wherein the upper chin support arm padding, the lower support arm padding, and the rear support member padding each having a laminated outer layer and a plurality of plurality of raised padding sections.

14. The adjustable cervical collar according to claim 13, wherein the plurality of raised padding sections being molded into a single layer.

15. The adjustable cervical collar according to claim 2, further comprising a safety lock coupled to the lower support arm, the cervical collar having a secured and unsecured configuration, the safety lock being configured to permit the cervical collar to toggle between the secured and unsecured configuration, wherein
the secured configuration prevents the cervical collar from assuming the unlocked configuration; and
the unsecured configuration permits the cervical collar to assume the unlocked configuration.

16. The adjustable collar according to claim 15, wherein
the safety lock comprises a blocking wall and a passthrough;
the locking mechanism release button comprises a locking extension, the locking extension being dimensionally smaller than the passthrough such that the locking extension can fit therewith.

17. The adjustable collar according to claim 16, wherein,
in the unsecured configuration, the locking extension is disposed within the passthrough; and
in the secured configuration, the blocking wall prevents lateral movement of the locking extension.

18. A method of adjusting a cervical collar comprising:
providing or receiving the cervical collar of claim 7;
depressing the locking mechanism release button, which pulls the elongated arm towards the front of the cervical collar; wherein
such forward progression of the elongated section causes forward progression of the arm engagement body coupled thereto;
such forward progression of the arm engagement body compresses the spring mechanism and permits lateral flexion of the latching tooth flexible arm;
lateral flexion of the latching tooth flexible arm disengages the latching tooth from the at least one groove between the plurality of teeth on the locking surface of the toothed sliding height adjustment piece to achieve the unlocked configuration; and
adjusting the lower support arm relative to the upper support arm.

19. The method of claim 18, further comprising:
releasing the locking mechanism release button to decompress the spring mechanism, wherein
decompression of the spring mechanism causes reward progression of the arm engagement body such that the arm engagement body prevents lateral flexion of the latching tooth flexible arm the latching tooth is reversibly engaged within at least one grove between the plurality of teeth of the locking surface and the cervical collar assumes a locked configuration.

20. The method of claim 18, wherein the cervical collar further comprises a safety lock coupled to the lower support arm the method further comprising twisting the safety lock to achieve an unsecured configuration prior to depressing the locking mechanism release button.
